(19) United States
(12) Patent Application Publication
(10) Pub. No.: US 2026/0256715 A1
(43) Pub. Date: Sep. 3, 2026

(54) PHARMACEUTICAL GEL PRODUCT

(71) Applicant: MOREMED INC., Toronto (CA)

(72) Inventors: Simon More, Toronto (CA); Khushwant Singh Bhullar, Moncton (CA)

(21) Appl. No.: 19/463,783

(22) Filed: Jan. 29, 2026

Related U.S. Application Data
(60) Provisional application No. 63/915,342, filed on Nov. 11, 2025.
(60) Provisional application No. 63/763,348, filed on Feb. 26, 2025.

(51) Int. Cl. A61K 31/135; A61J 1/06; A61J 7/00; A61K 9/00; A61K 9/06; A61K 36/736; A61K 47/10; A61K 47/12; A61K 47/22; A61K 47/36; A61K 47/46

(52) U.S. Cl. CPC A61K 31/135; A61J 1/067; A61J 7/0053; A61K 9/0053; A61K 9/06; A61K 36/736; A61K 47/10; A61K 47/12; A61K 47/22; A61K 47/36; A61K 47/46

(57) ABSTRACT

A single-use sleep aid includes a disposable gel storage and delivery device with a collapsible chamber and a delivery portion having a manually severable section for permanent unsealing. The chamber contains a single dose of 10–50 grams of gel with a viscosity of 600–1000 mPa·s at 25 °C. The gel includes diphenhydramine hydrochloride sleep aid (0.046–0.32 wt%), high methoxyl slow-set pectin (0.25–1.5 wt%) as a bioavailability enhancer and slow-release agent, Prunus cerasus extract (0.25–1.25 wt%) including a melatonin precursor, citric acid pectin activator (0.05–0.5 wt%) , honey (75–95 wt%), glycerin preservative (0.5–2.5 wt%), and ascorbic acid antioxidant and preservative (0.05–0.5 wt%). The collapsible chamber transitions from a first internal volume to a smaller second volume as the gel is delivered to the user’s mouth.

### FIELD OF THE INVENTION

This invention relates to pharmaceutical products, including sleep aids for promoting sleep and/or treating insomnia.

### BACKGROUND OF THE INVENTION

Insomnia is characterized by difficulty initiating sleep, staying asleep, and/or waking up too early, and is often accompanied by daytime drowsiness and impairments. Insomnia is the most common sleep disorder in the world, and impacts about 1 in 3 adults in the United States and about 10% of adults worldwide.

A variety of treatments are available for treating insomnia, including benzodiazepines, dual orexin receptor antagonists, sedating antidepressants, melatonin, and antihistamine drugs such as diphenhydramine and doxylamine.

### SUMMARY OF THE INVENTION

In one aspect the present invention provides a pharmaceutical product having a gel storage and delivery device carrying a consumable gel that contains a synthetic active pharmaceutical ingredient in an oral dosage form. The gel storage and delivery device is preferably a flexible pouch or other similar device that can be manually compressed in order to deliver the gel into a user’s mouth. In preferred embodiments, the gel may also contain a bioavailability enhancer that increases the bioavailability of the synthetic active pharmaceutical ingredient. The bioavailability enhancer may, for example, include a gelling agent such as pectin.

Pectin is found in the cell walls of terrestrial plants, and is widely used as a gelling agent and thickener in jams, jellies, and fruit preserves. Chemically, pectin is a long chain macromolecule polysaccharide composed of repeated galacturonic acid units, with varying degrees of methylation of carboxylic acid residues. Pectins with a low degree of methylation form gel in the presence of multivalent ions, while those with a higher degree of methylation form gel in acidic media with the addition of different sugars.

High methoxyl pectin, which has a degree of esterification that exceeds 50%, creates a three-dimensional cross-linked polymer network that remains stable in the acidic conditions of the human stomach. This enables protection of drugs from the harsh gastric environment and controlled release in the intestinal tract, enhancing bioavailability.

The inventors have appreciated that suitable gelling agents, such as pectin, can advantageously increase the bioavailability of active pharmaceutical ingredients, and that the resulting gel can be conveniently stored and administered to a user via a squeeze pack, a flexible pouch, or the like. The inventors have appreciated that a wide variety of different synthetic active pharmaceutical ingredients could be administered in this manner, including for example: anti-inflammatories, pain relievers, antihistamines, decongestants, cough suppressants, expectorants, weight loss medications, antidiarrheals, laxatives, anti-nausea medications, motion sickness medications, antidepressants, antipsychotics, anti-anxiety medications, addiction treatment medications, antiviral medications, antibiotics, statins, antihypertensive medications, synthetic hormones, antidiabetics, stomach acid reduction medications, contraceptives, diuretics, bronchodilators, anticonvulsants, synthetic steroids, anticoagulants, synthetic stimulants, non-steroidal anti-inflammatory drugs, analgesics, and/or antipyretics.

The inventors have further appreciated that gelling agents other than pectin could also be used to increase the bioavailability of active pharmaceutical ingredients, for example by protecting the active pharmaceutical ingredients from physical, chemical, and/or enzymatic degradation in the human digestive system. Other gelling agents that could be used include acacia, agar, methylcellulose, tragacanth, gelatin, collagen, guar gum, alginate, cornstarch, and/or chia seeds, for example.

In one particularly preferred embodiment, the invention provides a sleep aid in the form of a gel storage and delivery device that contains a gel for promoting sleep. The gel preferably contains pectin and diphenhydramine. The gel storage and delivery device is preferably a flexible pouch that can, for example, be squeezed in order to deliver the gel into a user’s mouth for ingestion.

The inventors have appreciated that the combination of pectin with diphenhydramine produces a gel that increases the bioavailability of the diphenhydramine. The inventors have further appreciated that the resulting gel can be quickly and efficiently ingested in its naturally settled form via a flexible pouch or other similar device.

Further aspects of the invention include:

A sleep aid, which optionally incorporates one or more features of any one or more of the preceding and/or following aspects, comprising: a gel storage and delivery device; and a gel comprising pectin and diphenhydramine; wherein the gel storage and delivery device comprises: a collapsible chamber that contains the gel; and a delivery portion for delivering the gel from the collapsible chamber to a user’s mouth; wherein the collapsible chamber collapses from an initial state having a first internal volume to a collapsed state having a second internal volume as the gel is delivered from the collapsible chamber to the user’s mouth, the first internal volume being greater than the second internal volume.

A sleep aid, which optionally incorporates one or more features of any one or more of the preceding and/or following aspects, wherein the gel storage and delivery device is configured to deliver the gel to the user’s mouth on application of a delivery force that creates a pressure differential between the collapsible chamber and the user’s mouth.

A sleep aid, which optionally incorporates one or more features of any one or more of the preceding and/or following aspects, wherein the delivery force comprises at least one of: a vacuum force applied by the user’s mouth to the delivery portion; and a compressive force applied manually to the collapsible chamber.

A sleep aid, which optionally incorporates one or more features of any one or more of the preceding and/or following aspects, wherein the gel storage and delivery device comprises a flexible pouch.

A sleep aid, which optionally incorporates one or more features of any one or more of the preceding and/or following aspects, wherein the flexible pouch has a base portion that is configured to engage with a horizontal support surface to support the flexible pouch in an upright orientation when the collapsible chamber is in the initial state.

A sleep aid, which optionally incorporates one or more features of any one or more of the preceding and/or following aspects, wherein the delivery portion is at an uppermost end of the flexible pouch when in the upright orientation.

A sleep aid, which optionally incorporates one or more features of any one or more of the preceding and/or following aspects, wherein the delivery portion comprises a severable section of the flexible pouch; wherein, when the severable section is intact, the delivery portion is sealed and the gel is prevented from exiting the flexible pouch; and wherein, when the severable section is ruptured, the delivery portion is open and the gel is able to be delivered from the flexible pouch.

A sleep aid, which optionally incorporates one or more features of any one or more of the preceding and/or following aspects, wherein the severable section is configured to be manually tearable.

A sleep aid, which optionally incorporates one or more features of any one or more of the preceding and/or following aspects, wherein the flexible pouch has a main pouch body with a pouch body width; and wherein the severable section has a narrower width than the main pouch body.

A sleep aid, which optionally incorporates one or more features of any one or more of the preceding and/or following aspects, wherein the pectin comprises methoxyl pectin.

A sleep aid, which optionally incorporates one or more features of any one or more of the preceding and/or following aspects, wherein the pectin comprises high methoxyl pectin.

A sleep aid, which optionally incorporates one or more features of any one or more of the preceding and/or following aspects, wherein the pectin comprises high methoxyl slow set pectin.

A sleep aid, which optionally incorporates one or more features of any one or more of the preceding and/or following aspects, wherein the diphenhydramine comprises diphenhydramine hydrochloride.

A sleep aid, which optionally incorporates one or more features of any one or more of the preceding and/or following aspects, wherein the gel storage and delivery device contains only one dose of the gel.

A sleep aid, which optionally incorporates one or more features of any one or more of the preceding and/or following aspects, wherein the gel storage and delivery device contains no more than 50 grams of the gel.

A sleep aid, which optionally incorporates one or more features of any one or more of the preceding and/or following aspects, wherein the gel storage and delivery device contains at least 10 grams of the gel.

A sleep aid, which optionally incorporates one or more features of any one or more of the preceding and/or following aspects, wherein the gel storage and delivery device contains between 25 grams and 40 grams of the gel.

A sleep aid, which optionally incorporates one or more features of any one or more of the preceding and/or following aspects, wherein the gel storage and delivery device contains about 32 grams of the gel.

A sleep aid, which optionally incorporates one or more features of any one or more of the preceding and/or following aspects, wherein the gel storage and delivery device contains no more than 100 mg of diphenhydramine.

A sleep aid, which optionally incorporates one or more features of any one or more of the preceding and/or following aspects, wherein the gel storage and delivery device contains at least 5 mg of diphenhydramine.

A sleep aid, which optionally incorporates one or more features of any one or more of the preceding and/or following aspects, wherein the gel storage and delivery device contains from 25 mg to 50 mg of diphenhydramine.

A sleep aid, which optionally incorporates one or more features of any one or more of the preceding and/or following aspects, wherein the gel storage and delivery device contains about 25 mg of diphenhydramine.

A sleep aid, which optionally incorporates one or more features of any one or more of the preceding and/or following aspects, wherein the gel storage and delivery device contains about 50 mg of diphenhydramine.

A sleep aid, which optionally incorporates one or more features of any one or more of the preceding and/or following aspects, wherein the gel storage and delivery device contains about 48 mg of diphenhydramine.

A sleep aid, which optionally incorporates one or more features of any one or more of the preceding and/or following aspects, wherein the gel contains no more than 1.5% pectin by weight.

A sleep aid, which optionally incorporates one or more features of any one or more of the preceding and/or following aspects, wherein the gel contains at least 0.25% pectin by weight.

A sleep aid, which optionally incorporates one or more features of any one or more of the preceding and/or following aspects, wherein the gel contains about 0.8% pectin by weight.

A sleep aid, which optionally incorporates one or more features of any one or more of the preceding and/or following aspects, wherein the gel contains about 0.5% pectin by weight.

A sleep aid, which optionally incorporates one or more features of any one or more of the preceding and/or following aspects, wherein the gel contains honey.

A sleep aid, which optionally incorporates one or more features of any one or more of the preceding and/or following aspects, wherein the gel contains at least 75% honey by weight.

A sleep aid, which optionally incorporates one or more features of any one or more of the preceding and/or following aspects, wherein the gel contains no more than 95% honey by weight.

A sleep aid, which optionally incorporates one or more features of any one or more of the preceding and/or following aspects, wherein the gel contains 85% to 90% honey by weight.

A sleep aid, which optionally incorporates one or more features of any one or more of the preceding and/or following aspects, wherein the gel contains tart cherry.

A sleep aid, which optionally incorporates one or more features of any one or more of the preceding and/or following aspects, wherein the gel contains about 0.5% tart cherry by weight.

A sleep aid, which optionally incorporates one or more features of any one or more of the preceding and/or following aspects, wherein the gel contains water.

A sleep aid, which optionally incorporates one or more features of any one or more of the preceding and/or following aspects, wherein the gel contains about 7.5% water by weight.

A sleep aid, which optionally incorporates one or more features of any one or more of the preceding and/or following aspects, wherein the gel contains glycerin.

A sleep aid, which optionally incorporates one or more features of any one or more of the preceding and/or following aspects, wherein the gel contains about 1.5% glycerin by weight.

A sleep aid, which optionally incorporates one or more features of any one or more of the preceding and/or following aspects, wherein the gel contains ascorbic acid.

A sleep aid, which optionally incorporates one or more features of any one or more of the preceding and/or following aspects, wherein the gel contains about 0.1% ascorbic acid by weight.

A sleep aid, which optionally incorporates one or more features of any one or more of the preceding and/or following aspects, wherein the gel contains citric acid.

A sleep aid, which optionally incorporates one or more features of any one or more of the preceding and/or following aspects, wherein the gel contains about 0.1% citric acid by weight.

A sleep aid, which optionally incorporates one or more features of any one or more of the preceding and/or following aspects, wherein the gel contains potassium sorbate.

A sleep aid, which optionally incorporates one or more features of any one or more of the preceding and/or following aspects, wherein the gel contains about 0.05% potassium sorbate by weight.

A sleep aid, which optionally incorporates one or more features of any one or more of the preceding and/or following aspects, wherein the gel contains cherry flavor.

A sleep aid, which optionally incorporates one or more features of any one or more of the preceding and/or following aspects, wherein the gel contains about 0.5% cherry flavor by weight.

A sleep aid, which optionally incorporates one or more features of any one or more of the preceding and/or following aspects, wherein the gel contains Piper longum.

A sleep aid, which optionally incorporates one or more features of any one or more of the preceding and/or following aspects, wherein the gel contains about 1% Piper longum by weight.

A sleep aid, which optionally incorporates one or more features of any one or more of the preceding and/or following aspects, wherein the gel contains Valeriana wallichii.

A sleep aid, which optionally incorporates one or more features of any one or more of the preceding and/or following aspects, wherein the gel contains about 0.5% Valeriana wallichii by weight.

A sleep aid, which optionally incorporates one or more features of any one or more of the preceding and/or following aspects, wherein the gel contains Melissa officinalis.

A sleep aid, which optionally incorporates one or more features of any one or more of the preceding and/or following aspects, wherein the gel contains about 0.75% Melissa officinalis by weight.

A sleep aid, which optionally incorporates one or more features of any one or more of the preceding and/or following aspects, wherein the gel contains Rosmarinus officinalis.

A sleep aid, which optionally incorporates one or more features of any one or more of the preceding and/or following aspects, wherein the gel contains about 0.75% Rosmarinus officinalis by weight.

A sleep aid, which optionally incorporates one or more features of any one or more of the preceding and/or following aspects, wherein the gel contains: about 0.046% to 0.32% diphenhydramine by weight; 0.25% to 1.5% pectin by weight; 0.25% to 1.25% tart cherry by weight; 75% to 95% honey by weight; 0.5% to 2.5 % glycerin by weight; 0.05% to 0.5% ascorbic acid by weight; 0.05% to 0.5% citric acid by weight; 0.05% to 0.5% potassium sorbate by weight; 0.1% to 1% cherry flavor by weight; 0.25% to 1.5% Piper longum by weight; 0.25% to 1.5% Valeriana wallichii by weight; 0.25% to 1.5% Melissa officinalis by weight; 0.25% to 1.5% Rosmarinus officinalis by weight; and 5% to 15% water by weight.

A sleep aid, which optionally incorporates one or more features of any one or more of the preceding and/or following aspects, wherein the gel contains: 0.15% diphenhydramine by weight; 0.8% high methoxyl slow set pectin by weight; 0.5% tart cherry by weight; 85.8% honey by weight; 1.5% glycerin by weight; 0.1% ascorbic acid by weight; 0.1% citric acid by weight; 0.05% potassium sorbate by weight; 0.5% cherry flavor by weight; 1% Piper longum by weight; 0.5% Valeriana wallichii by weight; 0.75% Melissa officinalis by weight; 0.75% Rosmarinus officinalis by weight; and 7.5% water by weight.

A sleep aid, which optionally incorporates one or more features of any one or more of the preceding and/or following aspects, wherein the gel contains: 0.15% diphenhydramine by weight; 0.5% high methoxyl slow set pectin by weight; 0.5% tart cherry by weight; 86.1% honey by weight; 1.5% glycerin by weight; 0.1% ascorbic acid by weight; 0.1% citric acid by weight; 0.05% potassium sorbate by weight; 0.5% cherry flavor by weight; 1% Piper longum by weight; 0.5% Valeriana wallichii by weight; 0.75% Melissa officinalis by weight; 0.75% Rosmarinus officinalis by weight; and 7.5% water by weight.

Use of the sleep aid in accordance with any one or more of the preceding aspects to promote sleep.

Use of the sleep aid in accordance with any one or more of the preceding aspects to treat insomnia.

A method, which optionally incorporates one or more features of any one or more of the preceding and/or following aspects, comprising: providing the sleep aid in accordance with any one or more of the preceding aspects; and instructing a user to ingest the gel in order to promote sleep.

A method, which optionally incorporates one or more features of any one or more of the preceding and/or following aspects, comprising: providing the sleep aid in accordance with any one or more of the preceding aspects; and instructing a user to ingest the gel in order to treat insomnia.

A method, which optionally incorporates one or more features of any one or more of the preceding and/or following aspects, comprising: obtaining the sleep aid in accordance with any one or more of the preceding aspects; and ingesting the gel in order to promote sleep.

A method, which optionally incorporates one or more features of any one or more of the preceding and/or following aspects, comprising: obtaining the sleep aid in accordance with any one or more of the preceding aspects; and ingesting the gel in order to treat insomnia.

A sleep aid, which optionally incorporates one or more features of any one or more of the preceding and/or following aspects, comprising: a gel storage and delivery device; and a gel containing a sleep promoting ingredient; wherein the gel storage and delivery device comprises: a collapsible chamber that contains the gel; and a delivery portion for delivering the gel from the collapsible chamber to a user’s mouth; wherein the collapsible chamber collapses from an initial state having a first internal volume to a collapsed state having a second internal volume as the gel is delivered from the collapsible chamber to the user’s mouth, the first internal volume being greater than the second internal volume.

A sleep aid, which optionally incorporates one or more features of any one or more of the preceding and/or following aspects, wherein the sleep promoting ingredient comprises diphenhydramine.

A sleep aid, which optionally incorporates one or more features of any one or more of the preceding and/or following aspects, wherein the gel contains pectin.

A product, which optionally incorporates one or more features of any one or more of the preceding and/or following aspects, comprising: a gel storage and delivery device; and a gel comprising pectin and diphenhydramine; wherein the gel storage and delivery device comprises: a collapsible chamber that contains the gel; and a delivery portion for delivering the gel from the collapsible chamber to a user’s mouth; wherein the collapsible chamber collapses from an initial state having a first internal volume to a collapsed state having a second internal volume as the gel is delivered from the collapsible chamber to the user’s mouth, the first internal volume being greater than the second internal volume.

A pharmaceutical product, which optionally incorporates one or more features of any one or more of the preceding and/or following aspects, comprising: a gel storage and delivery device; and a gel containing a synthetic active pharmaceutical ingredient; wherein the gel storage and delivery device comprises: a collapsible chamber that contains the gel; and a delivery portion for delivering the gel from the collapsible chamber to a user’s mouth; wherein the collapsible chamber collapses from an initial state having a first internal volume to a collapsed state having a second internal volume as the gel is delivered from the collapsible chamber to the user’s mouth, the first internal volume being greater than the second internal volume.

A pharmaceutical product, which optionally incorporates one or more features of any one or more of the preceding and/or following aspects, wherein the gel further comprises a bioavailability enhancer that increases the bioavailability of the synthetic active pharmaceutical ingredient.

A pharmaceutical product, which optionally incorporates one or more features of any one or more of the preceding and/or following aspects, wherein the bioavailability enhancer comprises a gelling agent.

A pharmaceutical product, which optionally incorporates one or more features of any one or more of the preceding and/or following aspects, wherein the bioavailability enhancer comprises a polymer.

A pharmaceutical product, which optionally incorporates one or more features of any one or more of the preceding and/or following aspects, wherein the bioavailability enhancer comprises at least one of: pectin, acacia, agar, methylcellulose, tragacanth, gelatin, collagen, guar gum, alginate, cornstarch, and chia seeds.

A pharmaceutical product, which optionally incorporates one or more features of any one or more of the preceding and/or following aspects, wherein the bioavailability enhancer comprises pectin.

A pharmaceutical product, which optionally incorporates one or more features of any one or more of the preceding and/or following aspects, wherein the pharmaceutical product is a sleep aid.

A pharmaceutical product, which optionally incorporates one or more features of any one or more of the preceding and/or following aspects, wherein the synthetic active pharmaceutical ingredient comprises a drug that promotes sleep.

A pharmaceutical product, which optionally incorporates one or more features of any one or more of the preceding and/or following aspects, wherein the synthetic active pharmaceutical ingredient comprises at least one of: diphenhydramine, cetirizine, trazodone, zolpidem, and fexofenadine.

A pharmaceutical product, which optionally incorporates one or more features of any one or more of the preceding and/or following aspects, wherein the synthetic active pharmaceutical ingredient comprises diphenhydramine.

A pharmaceutical product, which optionally incorporates one or more features of any one or more of the preceding and/or following aspects, wherein the gel storage and delivery device comprises a flexible pouch.

A pharmaceutical product, which optionally incorporates one or more features of any one or more of the preceding and/or following aspects, wherein the gel storage and delivery device comprises a squeeze pack.

A pharmaceutical product, which optionally incorporates one or more features of any one or more of the preceding and/or following aspects, wherein the synthetic active pharmaceutical ingredient comprises at least one of: an anti-inflammatory, a pain reliever, an antihistamine, a decongestant, a cough suppressant, an expectorant, a weight loss medication, an antidiarrheal, a laxative, an anti-nausea medication, a motion sickness medication, an antidepressant, an antipsychotic, an anti-anxiety medication, an addiction treatment medication, an antiviral medication, an antibiotic, a statin, an antihypertensive medication, a synthetic hormone, an antidiabetic, a stomach acid reduction medication, a contraceptive, a diuretic, a bronchodilator, an anticonvulsant, a synthetic steroid, an anticoagulant, a synthetic stimulant, a non-steroidal anti-inflammatory drug, an analgesic, and an antipyretic.

A pharmaceutical product, which optionally incorporates one or more features of any one or more of the preceding and/or following aspects, wherein the synthetic active pharmaceutical ingredient comprises ibuprofen.

A pharmaceutical product, which optionally incorporates one or more features of any one or more of the preceding and/or following aspects, wherein the synthetic active pharmaceutical ingredient comprises acetaminophen.

A pharmaceutical product, which optionally incorporates one or more features of any one or more of the preceding and/or following aspects, comprising: a storage and delivery device; and a fluid containing a synthetic active pharmaceutical ingredient; wherein the storage and delivery device comprises: a collapsible chamber that contains the fluid; and a delivery portion for delivering the fluid from the collapsible chamber to a user’s mouth; wherein the collapsible chamber collapses from an initial state having a first internal volume to a collapsed state having a second internal volume as the fluid is delivered from the collapsible chamber to the user’s mouth, the first internal volume being greater than the second internal volume.

A pharmaceutical product, which optionally incorporates one or more features of any one or more of the preceding and/or following aspects, wherein the fluid further comprises a bioavailability enhancer that increases the bioavailability of the synthetic active pharmaceutical ingredient.

A pharmaceutical product, which optionally incorporates one or more features of any one or more of the preceding and/or following aspects, wherein the bioavailability enhancer comprises a viscosity modifying agent that increases the viscosity of the fluid.

A pharmaceutical product, which optionally incorporates one or more features of any one or more of the preceding and/or following aspects, wherein the fluid further comprises a viscosity modifying agent that increases the viscosity of the fluid.

A pharmaceutical product, which optionally incorporates one or more features of any one or more of the preceding and/or following aspects, wherein the fluid comprises at least one of: a liquid, a dispersion, a foam, a paste, a Newtonian fluid, a non-Newtonian fluid, an emulsion, a suspension, and a colloid.

A pharmaceutical product, which optionally incorporates one or more features of any one or more of the preceding and/or following aspects, wherein the fluid has a viscosity greater than 1500 mPA·s.

A pharmaceutical product, which optionally incorporates one or more features of any one or more of the preceding and/or following aspects, wherein the fluid has a viscosity lower than 2500 mPA·s.

A pharmaceutical product, which optionally incorporates one or more features of any one or more of the preceding and/or following aspects, wherein the fluid has a viscosity from 1900 mPA·s to 2500 mPA·s.

A pharmaceutical product, which optionally incorporates one or more features of any one or more of the preceding and/or following aspects, wherein the gel has a viscosity greater than 1500 mPA·s.

A pharmaceutical product, which optionally incorporates one or more features of any one or more of the preceding and/or following aspects, wherein the gel has a viscosity lower than 2500 mPA·s.

A pharmaceutical product, which optionally incorporates one or more features of any one or more of the preceding and/or following aspects, wherein the gel has a viscosity from 1900 mPA·s to 2500 mPA·s.

A pharmaceutical product, which optionally incorporates one or more features of any one or more of the preceding and/or following aspects, comprising: a storage and delivery device; and a fluid containing an active pharmaceutical ingredient in an oral dosage form; wherein the storage and delivery device comprises: a collapsible chamber that contains the fluid; and a delivery portion for delivering the fluid from the collapsible chamber to a user’s mouth for ingestion; wherein the collapsible chamber collapses from an initial state having a first internal volume to a collapsed state having a second internal volume as the fluid is delivered from the collapsible chamber to the user’s mouth, the first internal volume being greater than the second internal volume.

A pharmaceutical product, which optionally incorporates one or more features of any one or more of the preceding and/or following aspects, wherein the active pharmaceutical ingredient comprises a synthetic active pharmaceutical ingredient.

A pharmaceutical product, which optionally incorporates one or more features of any one or more of the preceding and/or following aspects, wherein the fluid comprises a bioavailability enhancer that increases the bioavailability of the active pharmaceutical ingredient.

A pharmaceutical product, which optionally incorporates one or more features of any one or more of the preceding and/or following aspects, wherein the bioavailability enhancer comprises a gelling agent.

A pharmaceutical product, which optionally incorporates one or more features of any one or more of the preceding and/or following aspects, wherein the bioavailability enhancer comprises a polymer.

A pharmaceutical product, which optionally incorporates one or more features of any one or more of the preceding and/or following aspects, wherein the bioavailability enhancer comprises at least one of: pectin, acacia, agar, methylcellulose, tragacanth, gelatin, collagen, guar gum, alginate, cornstarch, and chia seeds.

A pharmaceutical product, which optionally incorporates one or more features of any one or more of the preceding and/or following aspects, wherein the fluid comprises pectin.

A pharmaceutical product, which optionally incorporates one or more features of any one or more of the preceding and/or following aspects, wherein the storage and delivery device comprises a flexible pouch.

A pharmaceutical product, which optionally incorporates one or more features of any one or more of the preceding and/or following aspects, wherein the storage and delivery device comprises a squeeze pack.

A pharmaceutical product, which optionally incorporates one or more features of any one or more of the preceding and/or following aspects, wherein the pharmaceutical product is a sleep aid.

A pharmaceutical product, which optionally incorporates one or more features of any one or more of the preceding and/or following aspects, wherein the active pharmaceutical ingredient comprises a drug that promotes sleep.

A pharmaceutical product, which optionally incorporates one or more features of any one or more of the preceding and/or following aspects, wherein active pharmaceutical ingredient comprises at least one of: diphenhydramine, cetirizine, trazodone, zolpidem, fexofenadine, doxylamine succinate, and promethazine.

A pharmaceutical product, which optionally incorporates one or more features of any one or more of the preceding and/or following aspects, wherein the active pharmaceutical ingredient comprises diphenhydramine.

A pharmaceutical product, which optionally incorporates one or more features of any one or more of the preceding and/or following aspects, wherein the fluid comprises tart cherry.

A pharmaceutical product, which optionally incorporates one or more features of any one or more of the preceding and/or following aspects, wherein the fluid comprises honey.

A pharmaceutical product, which optionally incorporates one or more features of any one or more of the preceding and/or following aspects, wherein the fluid comprises glycerin.

A pharmaceutical product, which optionally incorporates one or more features of any one or more of the preceding and/or following aspects, wherein the fluid comprises ascorbic acid.

A pharmaceutical product, which optionally incorporates one or more features of any one or more of the preceding and/or following aspects, wherein the fluid comprises citric acid.

A pharmaceutical product, which optionally incorporates one or more features of any one or more of the preceding and/or following aspects, wherein the fluid comprises cherry flavor.

A pharmaceutical product, which optionally incorporates one or more features of any one or more of the preceding and/or following aspects, wherein the fluid comprises water.

A pharmaceutical product, which optionally incorporates one or more features of any one or more of the preceding and/or following aspects, wherein the storage and delivery device contains about 32 grams of the fluid.

A pharmaceutical product, which optionally incorporates one or more features of any one or more of the preceding and/or following aspects, wherein the fluid contains 15 to 50 mg of diphenhydramine.

A pharmaceutical product, which optionally incorporates one or more features of any one or more of the preceding and/or following aspects, wherein the fluid contains 0.046 to 0.32% by weight of diphenhydramine.

A pharmaceutical product, which optionally incorporates one or more features of any one or more of the preceding and/or following aspects, wherein the fluid contains 60 to 260 mg of pectin.

A pharmaceutical product, which optionally incorporates one or more features of any one or more of the preceding and/or following aspects, wherein the fluid contains 0.18 to 0.81% by weight of pectin.

A pharmaceutical product, which optionally incorporates one or more features of any one or more of the preceding and/or following aspects, wherein the fluid contains 80 to 200 mg of tart cherry.

A pharmaceutical product, which optionally incorporates one or more features of any one or more of the preceding and/or following aspects, wherein the fluid contains 0.25 to 0.625% by weight of tart cherry.

A pharmaceutical product, which optionally incorporates one or more features of any one or more of the preceding and/or following aspects, wherein the fluid contains 16,000 to 30,400 mg of honey.

A pharmaceutical product, which optionally incorporates one or more features of any one or more of the preceding and/or following aspects, wherein the fluid contains 50 to 95% by weight of honey.

A pharmaceutical product, which optionally incorporates one or more features of any one or more of the preceding and/or following aspects, wherein the fluid contains 160 to 640 mg of glycerin.

A pharmaceutical product, which optionally incorporates one or more features of any one or more of the preceding and/or following aspects, wherein the fluid contains 0.5 to 2% by weight of glycerin.

A pharmaceutical product, which optionally incorporates one or more features of any one or more of the preceding and/or following aspects, wherein the fluid contains 16 to 128 mg of ascorbic acid.

A pharmaceutical product, which optionally incorporates one or more features of any one or more of the preceding and/or following aspects, wherein the fluid contains 0.05 to 0.4% by weight of ascorbic acid.

A pharmaceutical product, which optionally incorporates one or more features of any one or more of the preceding and/or following aspects, wherein the fluid contains 16 to 128 mg of citric acid.

A pharmaceutical product, which optionally incorporates one or more features of any one or more of the preceding and/or following aspects, wherein the fluid contains 0.05 to 0.4% by weight of citric acid.

A pharmaceutical product, which optionally incorporates one or more features of any one or more of the preceding and/or following aspects, wherein the fluid contains 32 to 320 mg of cherry flavor.

A pharmaceutical product, which optionally incorporates one or more features of any one or more of the preceding and/or following aspects, wherein the fluid contains 0.1 to 1.0% by weight of cherry flavor.

A pharmaceutical product, which optionally incorporates one or more features of any one or more of the preceding and/or following aspects, wherein the fluid contains 960 to 3200 mg of water.

A pharmaceutical product, which optionally incorporates one or more features of any one or more of the preceding and/or following aspects, wherein the fluid contains 3 to 10% by weight of water.

A pharmaceutical product, which optionally incorporates one or more features of any one or more of the preceding and/or following aspects, wherein the fluid contains: about 0.05 g or about 0.16% by weight of diphenhydramine hydrochloride; about 0.16 g or about 0.50% by weight of slow set high methoxyl pectin; about 0.16 g or about 0.50% by weight of tart cherry (Prunus Cerasus); about 28.5260 g or about 89.14% by weight of Manuka honey; about 0.48 g or about 1.50% by weight of glycerin; about 0.032 g or about 0.10% by weight of ascorbic acid; about 0.032 g or about 0.10% by weight of citric acid; about 0.16 g or about 0.50% by weight of natural cherry flavor; and about 2.4 g or about 7.50% by weight of purified water.

A pharmaceutical product, which optionally incorporates one or more features of any one or more of the preceding and/or following aspects, wherein the fluid contains sodium citrate.

A pharmaceutical product, which optionally incorporates one or more features of any one or more of the preceding and/or following aspects, wherein the fluid contains 8 to 64 mg of sodium citrate.

A pharmaceutical product, which optionally incorporates one or more features of any one or more of the preceding and/or following aspects, wherein the fluid contains 0.025 to 0.2% by weight of sodium citrate.

A pharmaceutical product, which optionally incorporates one or more features of any one or more of the preceding and/or following aspects, wherein the fluid contains: about 0.05 g or about 0.16% by weight of diphenhydramine hydrochloride; about 0.16 g or about 0.50% by weight of slow set high methoxyl pectin; about 0.16 g or about 0.50% by weight of tart cherry (Prunus Cerasus); about 28.494 g or about 89.04% by weight of Manuka honey; about 0.48 g or about 1.50% by weight of glycerin; about 0.032 g or about 0.10% by weight of ascorbic acid; about 0.032 g or about 0.10% by weight of citric acid; about 0.032 g or about 0.10% by weight of sodium citrate; about 0.16 g or about 0.50% by weight of natural cherry flavor; and about 2.4 g or about 7.50% by weight of purified water.

A pharmaceutical product, which optionally incorporates one or more features of any one or more of the preceding and/or following aspects, wherein the fluid is for treating pain.

A pharmaceutical product, which optionally incorporates one or more features of any one or more of the preceding and/or following aspects, wherein the fluid is for treating menstrual pain.

A pharmaceutical product, which optionally incorporates one or more features of any one or more of the preceding and/or following aspects, wherein the fluid is for treating headache.

A pharmaceutical product, which optionally incorporates one or more features of any one or more of the preceding and/or following aspects, wherein the fluid comprises at least one of: acetaminophen, ibuprofen, naproxen, aspirin, celecoxib, diclofenac, ketoprofen, mefenamic acid, magnesium salicylate, ketorolac, and excedrin.

A pharmaceutical product, which optionally incorporates one or more features of any one or more of the preceding and/or following aspects, wherein the fluid comprises acetaminophen.

A pharmaceutical product, which optionally incorporates one or more features of any one or more of the preceding and/or following aspects, wherein the fluid comprises pamabrom.

A pharmaceutical product, which optionally incorporates one or more features of any one or more of the preceding and/or following aspects, wherein the fluid comprises honey.

A pharmaceutical product, which optionally incorporates one or more features of any one or more of the preceding and/or following aspects, wherein the fluid comprises glycerin.

A pharmaceutical product, which optionally incorporates one or more features of any one or more of the preceding and/or following aspects, wherein the fluid comprises ascorbic acid.

A pharmaceutical product, which optionally incorporates one or more features of any one or more of the preceding and/or following aspects, wherein the fluid comprises citric acid.

A pharmaceutical product, which optionally incorporates one or more features of any one or more of the preceding and/or following aspects, wherein the fluid comprises strawberry flavor.

A pharmaceutical product, which optionally incorporates one or more features of any one or more of the preceding and/or following aspects, wherein the fluid comprises water.

A pharmaceutical product, which optionally incorporates one or more features of any one or more of the preceding and/or following aspects, wherein the storage and delivery device contains about 32 grams of the fluid.

A pharmaceutical product, which optionally incorporates one or more features of any one or more of the preceding and/or following aspects, wherein the fluid contains 220 to 600 mg of acetaminophen.

A pharmaceutical product, which optionally incorporates one or more features of any one or more of the preceding and/or following aspects, wherein the fluid contains 0.687 to 1.875% by weight of acetaminophen.

A pharmaceutical product, which optionally incorporates one or more features of any one or more of the preceding and/or following aspects, wherein the fluid contains 5 to 25 mg of pamabrom.

A pharmaceutical product, which optionally incorporates one or more features of any one or more of the preceding and/or following aspects, wherein the fluid contains 0.0156 to 0.078% by weight of pamabrom.

A pharmaceutical product, which optionally incorporates one or more features of any one or more of the preceding and/or following aspects, wherein the fluid contains 60 to 260 mg of pectin.

A pharmaceutical product, which optionally incorporates one or more features of any one or more of the preceding and/or following aspects, wherein the fluid contains 0.18 to 0.81% by weight of pectin.

A pharmaceutical product, which optionally incorporates one or more features of any one or more of the preceding and/or following aspects, wherein the fluid contains 16,000 to 30,400 mg of honey.

A pharmaceutical product, which optionally incorporates one or more features of any one or more of the preceding and/or following aspects, wherein the fluid contains 50 to 95% by weight of honey.

A pharmaceutical product, which optionally incorporates one or more features of any one or more of the preceding and/or following aspects, wherein the fluid contains 160 to 640 mg of glycerin.

A pharmaceutical product, which optionally incorporates one or more features of any one or more of the preceding and/or following aspects, wherein the fluid contains 0.5 to 2% by weight of glycerin.

A pharmaceutical product, which optionally incorporates one or more features of any one or more of the preceding and/or following aspects, wherein the fluid contains 16 to 128 mg of ascorbic acid.

A pharmaceutical product, which optionally incorporates one or more features of any one or more of the preceding and/or following aspects, wherein the fluid contains 0.05 to 0.4% by weight of ascorbic acid.

A pharmaceutical product, which optionally incorporates one or more features of any one or more of the preceding and/or following aspects, wherein the fluid contains 16 to 128 mg of citric acid.

A pharmaceutical product, which optionally incorporates one or more features of any one or more of the preceding and/or following aspects, wherein the fluid contains 0.05 to 0.4% by weight of citric acid.

A pharmaceutical product, which optionally incorporates one or more features of any one or more of the preceding and/or following aspects, wherein the fluid contains 32 to 320 mg of strawberry flavor.

A pharmaceutical product, which optionally incorporates one or more features of any one or more of the preceding and/or following aspects, wherein the fluid contains 0.1 to 1.0% by weight of strawberry flavor.

A pharmaceutical product, which optionally incorporates one or more features of any one or more of the preceding and/or following aspects, wherein the fluid contains 960 to 3200 mg of water.

A pharmaceutical product, which optionally incorporates one or more features of any one or more of the preceding and/or following aspects, wherein the fluid contains 3 to 10% by weight of water.

A pharmaceutical product, which optionally incorporates one or more features of any one or more of the preceding and/or following aspects, wherein the fluid contains: about 0.5 g or about 1.56% by weight of acetaminophen; about 0.025 g or about 0.08% by weight of pamabrom; about 0.16 g or about 0.50% by weight of slow set high methoxyl pectin; about 28.405 g or about 88.77% by weight of Manuka honey; about 0.48 g or about 1.50% by weight of glycerin; about 0.04 g or about 0.13% by weight of ascorbic acid; about 0.06 g or about 0.19% by weight of citric acid; about 0.1 g or about 0.31% by weight of natural strawberry flavor; and about 2.23 g or about 6.97% by weight of purified water.

A pharmaceutical product, which optionally incorporates one or more features of any one or more of the preceding and/or following aspects, wherein the fluid comprises sodium citrate.

A pharmaceutical product, which optionally incorporates one or more features of any one or more of the preceding and/or following aspects, wherein the fluid contains 8 to 64 mg of sodium citrate.

A pharmaceutical product, which optionally incorporates one or more features of any one or more of the preceding and/or following aspects, wherein the fluid contains 0.025 to 0.2% by weight of sodium citrate.

A pharmaceutical product, which optionally incorporates one or more features of any one or more of the preceding and/or following aspects, wherein the fluid contains: about 0.5 g or about 1.56% by weight of acetaminophen; about 0.025 g or about 0.08% by weight of pamabrom; about 0.16 g or about 0.50% by weight of slow set high methoxyl pectin; about 28.345 g or about 88.58% by weight of Manuka honey; about 0.48 g or about 1.50% by weight of glycerin; about 0.04 g or about 0.13% by weight of ascorbic acid; about 0.06 g or about 0.19% by weight of citric acid; about 0.06 g or about 0.19% by weight of sodium citrate; about 0.1 g or about 0.31% by weight of natural strawberry flavor; and about 2.23 g or about 6.97% by weight of purified water.

A pharmaceutical product, which optionally incorporates one or more features of any one or more of the preceding and/or following aspects, wherein the fluid contains: about 0.5 g or about 1.56% by weight of acetaminophen; about 0.16 g or about 0.50% by weight of slow set high methoxyl pectin; about 28.43 g or about 88.84% by weight of Manuka honey; about 0.48 g or about 1.50% by weight of glycerin; about 0.04 g or about 0.13% by weight of ascorbic acid; about 0.06 g or about 0.19% by weight of citric acid; about 0.1 g or about 0.31% by weight of natural strawberry flavor; and about 2.23 g or about 6.97% by weight of purified water.

A pharmaceutical product, which optionally incorporates one or more features of any one or more of the preceding and/or following aspects, wherein the fluid contains: about 0.5 g or about 1.56% by weight of acetaminophen; about 0.16 g or about 0.50% by weight of slow set high methoxyl pectin; about 28.43 g or about 88.84% by weight of Manuka honey; about 0.48 g or about 1.50% by weight of glycerin; about 0.04 g or about 0.13% by weight of ascorbic acid; about 0.06 g or about 0.19% by weight of citric acid; about 0.1 g or about 0.31% by weight of natural strawberry flavor; and about 2.23 g or about 6.97% by weight of purified water.

A pharmaceutical product, which optionally incorporates one or more features of any one or more of the preceding and/or following aspects, wherein the fluid contains: about 0.5 g or about 1.56% by weight of acetaminophen; about 0.16 g or about 0.50% by weight of slow set high methoxyl pectin; about 28.37 g or about 88.66% by weight of Manuka honey; about 0.48 g or about 1.50% by weight of glycerin; about 0.04 g or about 0.13% by weight of ascorbic acid; about 0.06 g or about 0.19% by weight of citric acid; about 0.06 g or about 0.19% by weight of sodium citrate; about 0.1 g or about 0.31% by weight of natural strawberry flavor; and about 2.23 g or about 6.97% by weight of purified water.

A pharmaceutical product, which optionally incorporates one or more features of any one or more of the preceding and/or following aspects, wherein the fluid is for treating nausea.

A pharmaceutical product, which optionally incorporates one or more features of any one or more of the preceding and/or following aspects, wherein the fluid is for treating upset stomach.

A pharmaceutical product, which optionally incorporates one or more features of any one or more of the preceding and/or following aspects, wherein the fluid comprises at least one of: bismuth subsalicylate, dimenhydrinate, meclizine hydrochloride, phosphorated carbohydrate, sodium citrate dehydrate, doxylamine succinate, and pyridoxine hydrochloride.

A pharmaceutical product, which optionally incorporates one or more features of any one or more of the preceding and/or following aspects, wherein the fluid has a viscosity of at least 300 mPa·s at 25 degrees Celsius.

A pharmaceutical product, which optionally incorporates one or more features of any one or more of the preceding and/or following aspects, wherein the fluid has a viscosity between 300 mPa·s and 2000 mPa·s at 25 degrees Celsius.

A pharmaceutical product, which optionally incorporates one or more features of any one or more of the preceding and/or following aspects, wherein the fluid has a viscosity of at least 600 mPa·s at 25 degrees Celsius.

A pharmaceutical product, which optionally incorporates one or more features of any one or more of the preceding and/or following aspects, wherein the fluid has a viscosity between 600 mPa·s and 1000 mPa·s at 25 degrees Celsius.

A pharmaceutical product, which optionally incorporates one or more features of any one or more of the preceding and/or following aspects, wherein the fluid comprises a gel.

A sleep aid, which optionally incorporates one or more features of any one or more of the preceding and/or following aspects, comprising: a storage and delivery device; and a fluid comprising pectin and diphenhydramine in an oral dosage form; wherein the storage and delivery device comprises: a collapsible chamber that contains the fluid; and a delivery portion for delivering the fluid from the collapsible chamber to a user’s mouth for ingestion; wherein the collapsible chamber collapses from an initial state having a first internal volume to a collapsed state having a second internal volume as the fluid is delivered from the collapsible chamber to the user’s mouth, the first internal volume being greater than the second internal volume.

A sleep aid, which optionally incorporates one or more features of any one or more of the preceding and/or following aspects, wherein the fluid has a viscosity of at least 300 mPa·s at 25 degrees Celsius.

A sleep aid, which optionally incorporates one or more features of any one or more of the preceding and/or following aspects, wherein the fluid comprises a gel.

### BRIEF DESCRIPTION OF THE DRAWINGS

Further aspects and advantages of the invention will appear from the following description taken together with the accompanying drawings, in which:

FIG. 1 is a front view of a sleep aid in accordance with a preferred embodiment of the present invention;

FIG. 2 is a front view of the sleep aid shown in FIG. 1 in an opened state;

FIG. 3 is a bottom view of the sleep aid shown in FIG. 1 in an initial state;

FIG. 4 is a bottom view of the sleep aid shown in FIG. 1 in a collapsed state; and

FIG. 5 is a bar graph showing the results of a study on the effects of diphenhydramine and pectin on the mRNA expression of histidine decarboxylase.

### DETAILED DESCRIPTION OF THE DRAWINGS

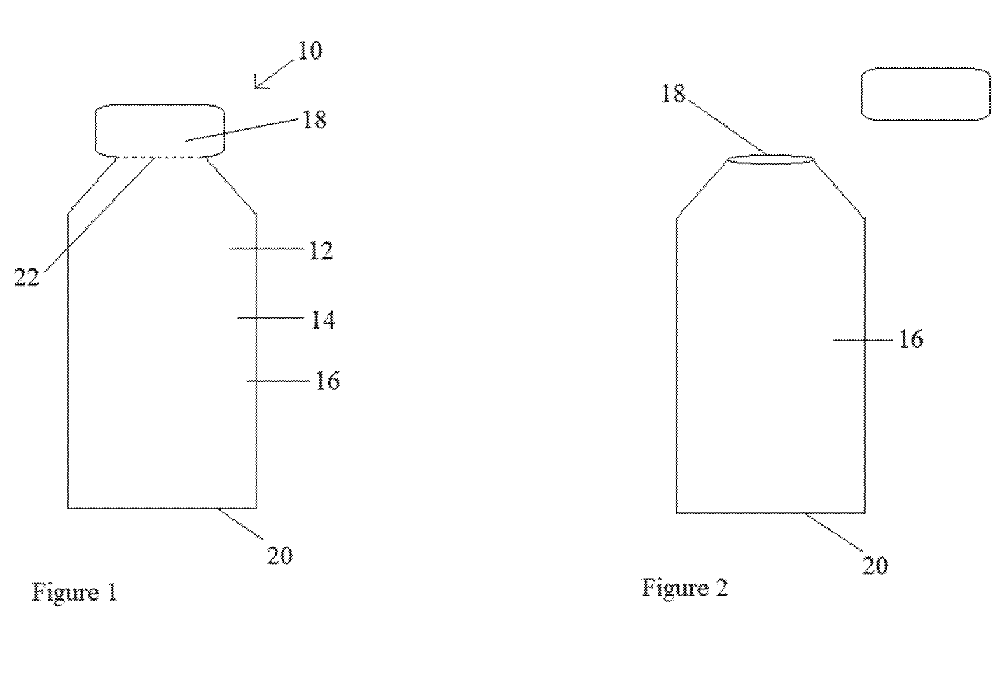

FIGS. 1 to 4 show a sleep aid 10 in accordance with a preferred embodiment of the present invention. The sleep aid 10 has a gel storage and delivery device 12 that contains a sleep promoting gel. The gel storage and delivery device 12 is a flexible pouch 14 having a collapsible chamber 16 that contains the gel, and a delivery portion 18 for delivering the gel from the collapsible chamber 16 to a user’s mouth. In some embodiments, sleep aid 10 is single-use and disposable. That is, the gel storage and delivery device 12 may be openable but not closeable. Additionally, its contents (gel) may be consumable but the gel storage and delivery device 12 may be generally non-refillable.

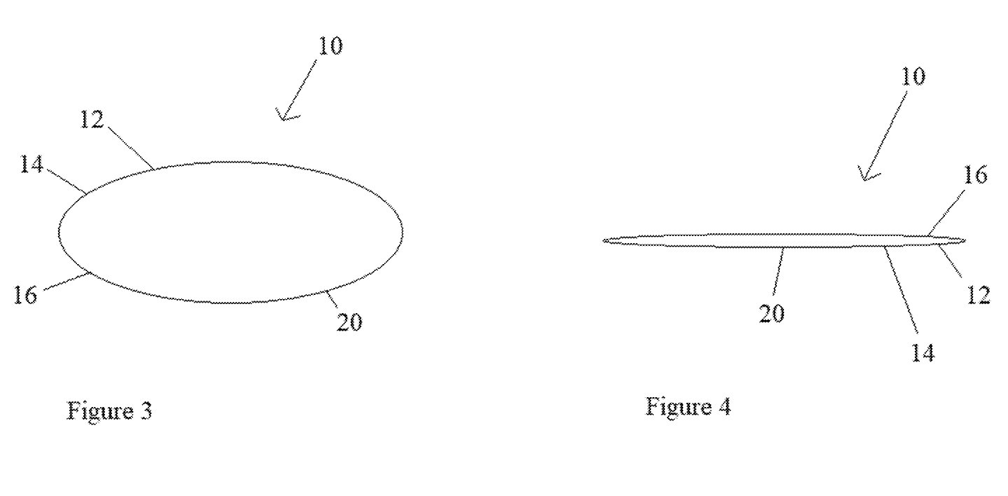

As can be seen in FIG. 3, the pouch 14 has a base portion 20 that has a generally elliptical shape when viewed from below. When the pouch 14 is in an initial state in which the pouch 14 is filled with the gel, the base portion 20 is configured to engage with a horizontal support surface, such as a table top, to support the pouch 14 in an upright orientation. As can be seen in FIG. 1, the delivery portion 18 is at the uppermost end of the pouch 14 when the pouch 14 is in the upright orientation.

The delivery portion 18 has a severable section 22, which is shown in dotted lines in FIG. 1. The severable section 22 is configured to be manually tearable. When the severed section 22 is intact as in FIG. 1, the delivery portion 18 is sealed and the gel is prevented from exiting the flexible pouch 14. When the severable section 22 is ruptured, as shown in FIG. 2, the delivery portion 18 is open and permanently unsealed, and the gel is able to be delivered from the flexible pouch 14. Preferably, the severable section 22 has a narrower width than the rest of the pouch body. This preferably makes it easier to sever the severable section 22, and also produces an opening that can more easily fit into the user’s mouth. Once severable section 22 is severed, sleep aid 10 has no intrinsic manner to reseal delivery portion 18.

The pouch 14 is configured to deliver the gel to a user’s mouth on application of a delivery force that creates a pressure differential between the collapsible chamber 16 and the user’s mouth. The delivery force may, for example, include a vacuum that is applied by the user’s mouth to the delivery portion 18. The delivery force could also include a compressive force applied to the collapsible chamber 16, for example by the user manually squeezing the pouch 14.

As the gel is delivered from the pouch 14 to the user’s mouth, the pouch 14 collapses from the initial state shown in FIG. 3 to the collapsed state shown in FIG. 4. The internal volume of the collapsible chamber 16 is greater when in the initial state than when in the collapsed state.

The gel preferably includes pectin and diphenhydramine. The pectin is preferably methoxyl pectin, and more preferably high methoxyl pectin, and even more preferably high methoxyl slow set pectin (HM SS pectin) with a degree of methylation greater than 50%. The diphenhydramine is preferably diphenhydramine hydrochloride.

The pouch 14 is preferably designed for a single use, and contains only one dose of the gel. Preferably, the pouch 14 contains from 10 grams to 50 grams of the gel; more preferably from 25 grams to 40 grams of the gel. In one particularly preferred embodiment, the pouch 14 contains about 32 grams of the gel. The pouch 14 preferably contains one adult dose of the gel. In other embodiments, the pouch 14 could contain a pediatric dose of the gel.

The pouch 14 preferably contains from 5 mg to 100 mg of diphenhydramine; more preferably from 25 mg to 50 mg of diphenhydramine. In some preferred embodiments, the pouch 14 contains about 25 mg of diphenhydramine. In other preferred embodiments, the pouch 14 contains about 50 mg of diphenhydramine. In some preferred embodiments, the pouch 14 contains about 48 mg of diphenhydramine. In some embodiments, pouch 14 contains no more than about 50 mg of diphenhydramine which may be the maximum allowable single dose according to regulations in some jurisdictions.

In some embodiments the gel contains from 0.25% to 1.5% pectin by weight. In some embodiments, the gel contains about 0.5% pectin by weight or about 0.8% pectin by weight, or between 0.5% and 0.8% pectin by weight.

The gel may also include one or more of the following additional ingredients: honey (preferably organic Manuka honey), tart cherry (Prunus cerasus), water (preferably nanopure water), glycerin (preferably from organic coconut oil), ascorbic acid, citric acid, potassium sorbate, cherry flavor, Piper longum, Valeriana wallichii, Melissa officinalis, and Rosmarinus officinalis. In some embodiments the gel includes all of the aforementioned ingredients.

In some embodiments of the invention, the gel contains 0.15% diphenhydramine by weight.

In some embodiments of the invention, the gel contains 0.25% to 1.5% pectin by weight, preferably high methoxyl slow set pectin.

In some embodiments of the invention, the gel contains 0.25% to 1.25% tart cherry by weight.

In some embodiments of the invention, the gel contains 75% to 95% honey by weight.

In some embodiments of the invention, the gel contains 0.5% to 2.5 % glycerin by weight.

In some embodiments of the invention, the gel contains 0.05% to 0.5% ascorbic acid by weight.

In some embodiments of the invention, the gel contains 0.05% to 0.5% citric acid by weight.

In some embodiments of the invention, the gel contains 0.05% to 0.5% potassium sorbate by weight.

In some embodiments of the invention, the gel contains 0.1% to 1% cherry flavor by weight.

In some embodiments of the invention, the gel contains 0.25% to 1.5% Piper longum by weight.

In some embodiments of the invention, the gel contains 0.25% to 1.5% Valeriana wallichii by weight.

In some embodiments of the invention, the gel contains 0.25% to 1.5% Melissa officinalis by weight.

In some embodiments of the invention, the gel contains 0.25% to 1.5% Rosmarinus officinalis by weight.

In some embodiments of the invention, the gel contains 5% to 15% water by weight.

In one embodiment of the invention, the gel contains: 0.15% (48 mg) diphenhydramine; 0.8% (256 mg) high methoxyl slow set pectin; 0.3% (96 mg) tart cherry; 87.25% (27920 mg) honey; 1.5% (480 mg) glycerin; 0.1% (32 mg) ascorbic acid; 0.1% (32 mg) citric acid; 0.05% (16 mg) potassium sorbate; 0.5% (160 mg) cherry flavor; 0.5% (160 mg) Piper longum; 0.25% (80 mg) Valeriana wallichii; 0.5% (160 mg) Melissa officinalis; 0.5% (160 mg) Rosmarinus officinalis; and 7.5% (2400 mg) water.

In one embodiment of the invention, the gel contains: 0.15% (48 mg) diphenhydramine; 0.5% (160 mg) high methoxyl slow set pectin; 0.5% (160 mg) tart cherry; 87.35% (27952 mg) honey; 1.5% (480 mg) glycerin; 0.1% (32 mg) ascorbic acid; 0.1% (32 mg) citric acid; 0.05% (16 mg) potassium sorbate; 0.5% (160 mg) cherry flavor; 0.5% (160 mg) Piper longum; 0.25% (80 mg) Valeriana wallichii; 0.5% (160 mg) Melissa officinalis; 0.5% (160 mg) Rosmarinus officinalis; and 7.5% (2400 mg) water.

In one embodiment, the gel contains: 0.15% (48 mg) diphenhydramine; 0.8% (256 mg) high methoxyl slow set pectin; 0.5% (160 mg) tart cherry; 85.8% (27456 mg) honey; 1.5% (480 mg) glycerin; 0.1% (32 mg) ascorbic acid; 0.1% (32 mg) citric acid; 0.05% (16 mg) potassium sorbate; 0.5% (160 mg) cherry flavor; 1% (320 mg) Piper longum; 0.5% (160 mg) Valeriana wallichii; 0.75% (240 mg) Melissa officinalis; 0.75% (240 mg) Rosmarinus officinalis; and 7.5% (2400 mg) water.

In another embodiment, the gel contains: 0.15% (48 mg) diphenhydramine; 0.5% (160 mg) high methoxyl slow set pectin; 0.5% (160 mg) tart cherry; 86.1% (27552 mg) honey; 1.5% (480 mg) glycerin; 0.1% (32 mg) ascorbic acid; 0.1% (32 mg) citric acid; 0.05% (16 mg) potassium sorbate; 0.5% (160 mg) cherry flavor; 1% (320 mg) Piper longum; 0.5% (160 mg) Valeriana wallichii; 0.75% (240 mg) Melissa officinalis; 0.75% (240 mg) Rosmarinus officinalis; and 7.5% (2400 mg) water.

Preferably, the ascorbic acid provides an antioxidant effect, and acts as a preservative.

Citric acid preferably acts as a flavor enhancer, preservative, and as an antioxidant. Citric acid also preferably helps in activating pectin activity (for this reason, citric acid may be referred to as a pectin activator), and aids in increasing the degree of pectin esterification.

Potassium sorbate preferably helps increase shelf life and protects against microbial deterioration (as used herein, potassium sorbate may be referred to as an antimicrobial preservative).

Cherry flavor preferably improves the flavor of the gel.

The Piper longum, Valeriana wallichii, Melissa officinalis, and Rosmarinus officinalis are preferably in the form of a herbal tincture that provides a unique tart flavor.

The honey preferably facilitates easier consumption of the product and helps to mask the taste of the diphenhydramine. Honey is also a natural sugar that contains essential nutrients, including trace vitamins and minerals and bioactive enzymes.

Pectin exhibits excellent gelling properties, and has a wide range of biological activities. Pectin may, for example, have wound healing, anti-cancer, anti-oxidation, anti-diabetic, and anti-inflammatory effects, as well as lowering cholesterol and improving gastrointestinal motility.

Tart cherry contains tryptophan (which is a precursor to melatonin), contains antioxidants, and is a source of potassium (which is important for regulating blood pressure).

Organic honey also contains tryptophan, and supports glycogen replenishment (which is important for liver function and brain activity).

Pectin contains soluble fiber, and improves cholesterol metabolism. Honey and ascorbic acid also help to activate pectin for gel formation. Methyl functional groups in the pectin chain make the active pharmaceutical ingredient (e.g. diphenhydramine) resistant to upper gastrointestinal enzyme degradation, and it is instead processed slowly by the lower gastrointestinal microbiota. Pectin helps to reduce physical, chemical, and/or enzymatic disruption of the active pharmaceutical ingredient (e.g. diphenhydramine).

Glycerin preferably helps to preserve the product.

Piper longum is used in traditional medicine to treat insomnia and improve sleep quality, and has been found to be a positive allosteric modulator of GABA_{A} receptors, which are involved in sleep regulation.

Valeriana wallichii acts on GABA receptors, promoting calmness and improving sleep quality, and is used for treating insomnia and anxiety.

Melissa officinalis is used to reduce anxiety and stress, and to improve sleep quality.

Rosmarinus officinalis is used as a traditional remedy for insomnia, depression and anxiety, and is rich in antioxidants and anti-inflammatory compounds. The herbal tincture preferably adds flavor and enhances the properties of the active pharmaceutical ingredient (e.g. diphenhydramine).

Water is preferably added to dissolve pectin and provide a base for jelly formation.

A method of using the sleep aid 10 will now be described with reference to the drawings. The sleep aid 10 is initially in the sealed state as shown in FIG. 1, and can be conveniently stored for example in a medicine cabinet, or on a bedside table in the upright orientation shown in FIG. 1. The sleep aid 10 is also conveniently portable, and can be carried for example in a purse, bag or pocket.

To use the sleep aid 10, the user first tears the severable section 22, which opens the delivery portion 18. The open delivery portion 18 is then placed in the user’s mouth and a delivery force is applied to create a pressure differential between the collapsible chamber 16 and the user’s mouth. The delivery force may, for example, include sucking on the delivery portion 18 to generate a vacuum and/or manually squeezing the pouch 14. The resulting pressure differential forces the gel from the pouch 14 into the user’s mouth, where it is then ingested by the user. As the gel exits the pouch 14, the pouch 14 collapses from the initial state shown in FIG. 3 to the collapsed state shown in FIG. 4. The empty pouch 14 can then be disposed of, and is preferably recycled.

The active ingredient in the gel, diphenhydramine, preferably helps the user to fall asleep. The pectin furthermore helps to increase the bioavailability of the diphenhydramine, and also provides for a controlled release of the diphenhydramine over time. This preferably helps the user stay asleep throughout the night. Accordingly, pectin may be referred to as a bioavailability enhancer and slow release agent for diphenhydramine.

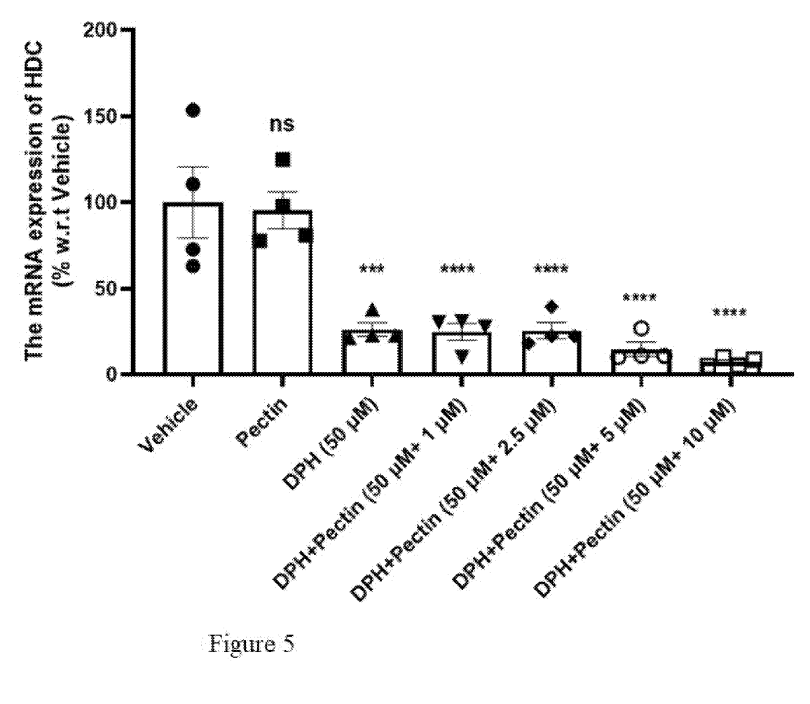

The inventors have conducted a study to further investigate the effects of pectin on the bioavailability of diphenhydramine. The results of the study are shown in FIG. 5 and summarized below.

Previous research has shown that antihistamines suppress the expression of histidine decarboxylase (HDC) mRNA expression. Mechanistically, antihistamines act as inverse agonists to inhibit histamine signaling by blocking histamine H1 receptor (H1R) or suppressing H1R signaling. Since HDC is the only enzyme that catalyzes histamine synthesis, HDC gene expression is an important biomarker for development of antihistamine drug candidates.

The inventors have conducted an experiment to measure the mRNA expression of HDC in Caco-2 cells following treatment with: (1) water saline solution; (2) pectin; (3) 50 μM of diphenhydramine; (4) 50 μM of diphenhydramine plus 1 μM of pectin; (5) 50 μM of diphenhydramine plus 2.5 μM of pectin; (6) 50 μM of diphenhydramine plus 5 μM of pectin; and (7) 50 μM of diphenhydramine plus 10 μM of pectin.

As can be seen in FIG. 5, the mRNA expression of HDC decreased when the cells were treated with diphenhydramine and with a combination of diphenhydramine and pectin. This reduction in the expression of HDC was further enhanced as the quantity of pectin increased.

It will be understood that, although various features of the invention have been described with respect to one or another of the embodiments of the invention, the various features and embodiments of the invention may be combined or used in conjunction with other features and embodiments of the invention as described and illustrated herein.

The invention is not limited to the particular preferred embodiments that have been shown in the drawings. Rather, any functionally and/or structurally equivalent alternatives could be used.

For example, the delivery portion 18 could incorporate a twistable cap instead of the severable section 22 as shown in the drawings. The gel storage and delivery device 12 is also not limited to being in the form of a pouch 14, and could alternatively have any other structure suitable for delivering the gel to a user, such as a tube or a syringe. The use of a flexible pouch 14 or squeeze pack is especially preferred, and preferably has a number of advantages such as being lightweight, self-standing, easy to store, cost-effective manufacturing, convenience, recyclable and easy to use.

The gel is also not limited to the specific combination of ingredients listed in preferred embodiments. For example, one or more of the following ingredients could be omitted from the gel: honey, tart cherry, water, glycerin, ascorbic acid, citric acid, potassium sorbate, cherry flavor, Piper longum, Valeriana wallichii, Melissa officinalis, and Rosmarinus officinalis. Other ingredients not specifically mentioned herein could also be incorporated into the gel. Although the diphenhydramine is preferably in the form of diphenhydramine hydrochloride, other suitable forms such as diphenhydramine citrate could also be used. Although the pectin is preferably in the form of high methoxyl pectin, other less preferred alternatives such as low methoxyl pectin could also be used.

In some embodiments of the invention, the diphenhydramine could be replaced or used in combination with another active pharmaceutical ingredient, such as cetirizine, trazodone, zolpidem, and/or fexofenadine.

In some embodiments of the invention, the pectin could be replaced or used in combination with another gelling and/or thickening agent, such as cornstarch, gelatin, agar, and/or chia seeds.

In some embodiments of the invention, Manuka honey could be replaced or used in combination with another product such as heather honey, Norwegian mountain blossom honey, pasteurized honey, buckwheat honey, raw honey, and/or maple syrup.

In some embodiments of the invention, the tart cherry could be replaced or used in combination with another product, such as cherry juice, black cherry extract, pineapple, dried cranberry, cranberry extract, and/or peach extract.

In some embodiments of the invention, the glycerin could be replaced or used in combination with another product, such as diethylene glycol, propylene glycol, ceramides, oils, and/or butter.

In some embodiments of the invention, the ascorbic acid could be replaced or used in combination with another product, such as vitamin C, Moringa oleifera, niacinamide, turmeric extract, green tea extract, orange extract, kiwi extract, and/or lemon extract.

In some embodiments of the invention, the citric acid could be replaced or used in combination with another product, such as white vinegar, lime juice or extract, orange juice or extract, apple cider vinegar, and/or lemon extract.

In some embodiments of the invention, the potassium sorbate could be replaced or used in combination with another product, such as potassium metabisulfite and/or citric acid.

In some embodiments of the invention, the cherry flavor could be replaced or used in combination with another product, such as cherry juice, cherry extract, and/or cherry powder.

In some embodiments of the invention, the Piper longum, Valeriana wallichii, Melissa officinalis, and Rosmarinus officinalis could be in powder form.

Preferably, the gel has a viscosity of 2500 mPA·s to 1500 mPA·s, and more preferably from 2300 mPA·s to 1900 mPA·s. In some preferred embodiments, the gel contains about 0.8% pectin by weight, and has a viscosity of 2200 mPA·s to 2300 mPA·s. In other preferred embodiments, the gel contains about 0.5% pectin by weight, and has a viscosity of 1900 mPA·s to 2000 mPA·s.

Preferably, the gel has a water activity that is less than 0.7. The gel also preferably has a water activity that is greater than 6. In some preferred embodiments, the water activity is between 0.69 and 0.64.

Preferably, the gel has a pH that is less than 4.5. The gel also preferably has a pH that is greater than 3.5. In some preferred embodiments, the pH is between 4.1 and 3.9, or the pH is about 4.

The gel is preferably an H1 antihistamine inhibition-based gel that is non-habit forming, has no added processed sugar, makes minimal use of chemicals and preservatives, and has an antioxidant rich and safe profile.

In some embodiments of the invention, the product is a consumable consumer packaged good. In some embodiments the product may be sold over the counter, or alternatively may require a prescription.

In some embodiments of the invention, the gel is for promoting sleep and comprises diphenhydramine, pectin, tart cherry, honey, glycerin, ascorbic acid, citric acid, cherry flavor, and water. The diphenhydramine is preferably diphenhydramine HCL. The pectin is preferably slow set high methoxyl pectin. The tart cherry is preferably Prunus Cerasus. The honey is preferably Manuka honey. The cherry flavor is preferably natural cherry flavor. The water is preferably purified water. The gel storage and delivery device preferably contains 32 grams of the gel. The gel preferably contains 15-100 mg or 0.046-0.32% by weight of diphenhydramine; 60-260 mg or 0.18-0.81% by weight of pectin; 80-200 mg or 0.25-0.625% by weight of tart cherry; 16,000-30,400 mg or 50-95% by weight of honey; 160-640 mg or 0.5-2% by weight of glycerin; 16-128 mg or 0.05-0.4% by weight of ascorbic acid; 16-128 mg or 0.05-0.4% by weight of citric acid; 32-320 mg or 0.1-1.0% by weight of cherry flavor; and 960-3200 mg or 3-10% by weight of water. In one preferred embodiment, the gel contains 0.05 g or 0.16% by weight of diphenhydramine HCL; 0.16 g or 0.50% by weight of slow set high methoxyl pectin; 0.16 g or 0.50% by weight of tart cherry (Prunus Cerasus); 28.5260 g or 89.14% by weight of Manuka honey; 0.48 g or 1.50% by weight of glycerin; 0.032 g or 0.10% by weight of ascorbic acid; 0.032 g or 0.10% by weight of citric acid; 0.16 g or 0.50% by weight of natural cherry flavor; and 2.4 g or 7.50% by weight of purified water.

In other embodiments of the invention, the gel for promoting sleep could include other active pharmaceutical ingredients such as doxylamine succinate and/or promethazine. The doxylamine succinate or promethazine may be provided in a dosage of 25 mg once daily, for example.

Other herbal aids such as valerian root, chamomile, lavender, passionflower, lemon balm, and/or hops could also be incorporated into the sleep promoting gel.

In some embodiments of the invention, the gel may incorporate a pH buffer such as sodium citrate. For example, the sleep promoting gel may include diphenhydramine, pectin, tart cherry, honey, glycerin, ascorbic acid, citric acid, sodium citrate, cherry flavor, and water. The diphenhydramine is preferably diphenhydramine HCL. The pectin is preferably slow set high methoxyl pectin. The tart cherry is preferably Prunus Cerasus. The honey is preferably Manuka honey. The cherry flavor is preferably natural cherry flavor. The water is preferably purified water. The gel storage and delivery device preferably contains 32 grams of the gel. The gel preferably contains 15-50 mg or 0.046-0.16% by weight of diphenhydramine; 60-260 mg or 0.18-0.81% by weight of pectin; 80-200 mg or 0.25-0.625% by weight of tart cherry; 16,000-30,400 mg or 50-95% by weight of honey; 160-640 mg or 0.5-2% by weight of glycerin; 16-128 mg or 0.05-0.4% by weight of glycerin; 16-128 mg or 0.05-0.4% by weight of citric acid; 8-64 mg or 0.025-0.2% by weight of sodium citrate; 32-320 mg or 0.1-1.0% by weight of cherry flavor; and 960-3200 mg or 3-10% by weight of water. In one preferred embodiment, the gel contains 0.05 g or 0.16% by weight of diphenhydramine HCL; 0.16 g or 0.50% by weight of slow set high methoxyl pectin; 0.16 g or 0.50% by weight of tart cherry (Prunus Cerasus); 28.494 g or 89.04% by weight of Manuka honey; 0.48 g or 1.50% by weight of glycerin; 0.032 g or 0.10% by weight of ascorbic acid; 0.032 g or 0.10% by weight of citric acid; 0.032 g or 0.10% by weight of sodium citrate; 0.16 g or 0.50% by weight of natural cherry flavor; and 2.4 g or 7.50% by weight of purified water.

In some embodiments of the invention, the gel is for treating pain. The gel may include a painkiller such as acetaminophen. In some embodiments, the gel is for treating menstrual pain. The gel for treating menstrual pain may include acetaminophen, pamabrom, pectin, honey, glycerin, ascorbic acid, citric acid, strawberry flavor, and water. The pectin is preferably slow set high methoxyl pectin. The honey is preferably Manuka honey. The strawberry flavor is preferably natural strawberry flavor. The water is preferably purified water. The gel storage and delivery device preferably contains 32 grams of the gel. The gel preferably contains 220-600 mg or 0.687-1.875% by weight of acetaminophen; 5-25 mg or 0.0156-0.078% by weight of pamabrom; 60-260 mg or 0.18-0.81% by weight of pectin; 16,000-30,400 mg or 50-95% by weight of honey; 160-640 mg or 0.5-2% by weight of glycerin; 16-128 mg or 0.05-0.4% by weight of ascorbic acid; 16-128 mg or 0.05-0.4% by weight of citric acid; 32-320 mg or 0.1-1.0% by weight of strawberry flavor; and 960-3200 mg or 3-10% by weight of water. In one preferred embodiment, the gel contains 0.5 g or 1.56% by weight of acetaminophen; 0.025 g or 0.08% by weight of pamabrom; 0.16 g or 0.50% by weight of slow set high methoxyl pectin; 28.405 g or 88.77% by weight of Manuka honey; 0.48 g or 1.50% by weight of glycerin; 0.04 g or 0.13% by weight of ascorbic acid; 0.06 g or 0.19% by weight of citric acid; 0.1 g or 0.31% by weight of natural strawberry flavor; and 2.23 g or 6.97% by weight of purified water.

In other embodiments of the invention, the gel for treating menstrual pain may include other active pharmaceutical ingredients such as ibuprofen, naproxen, aspirin, celecoxib, diclofenac, ketoprofen, mefenamic acid, and/or magnesium salicylate.

Other herbal additions such as ginger, chamomile, cramp bark, raspberry leaf, fennel and/or cinnamon could also be added as excipients.

Optionally, the gel may incorporate a pH buffer such as sodium citrate. For example, the gel for treating menstrual pain may include acetaminophen, pamabrom, pectin, honey, glycerin, ascorbic acid, citric acid, sodium citrate, strawberry flavor, and water. The pectin is preferably slow set high methoxyl pectin. The honey is preferably Manuka honey. The strawberry flavor is preferably natural strawberry flavor. The water is preferably purified water. The gel storage and delivery device preferably contains 32 grams of the gel. The gel preferably contains 220-600 mg or 0.687-1.875% by weight of acetaminophen; 5-25 mg or 0.0156-0.078% by weight of pamabrom; 60-260 mg or 0.18-0.81% by weight of pectin; 16,000-30,400 mg or 50-95% by weight of honey; 160-640 mg or 0.5-2% by weight of glycerin; 16-128 mg or 0.05-0.4% by weight of ascorbic acid; 16-128 mg or 0.05-0.4% by weight of citric acid; 8-64 mg or 0.025-0.2% by weight of sodium citrate; 32-320 mg or 0.1-1.0% by weight of strawberry flavor; and 960-3200 mg or 3-10% by weight of water. In one preferred embodiment, the gel contains 0.5 g or 1.56% by weight of acetaminophen; 0.025 g or 0.08% by weight of pamabrom; 0.16 g or 0.50% by weight of slow set high methoxyl pectin; 28.345 g or 88.58% by weight of Manuka honey; 0.48 g or 1.50% by weight of glycerin; 0.04 g or 0.13% by weight of ascorbic acid; 0.06 g or 0.19% by weight of citric acid; 0.06 g or 0.19% by weight of sodium citrate; 0.1 g or 0.31% by weight of natural strawberry flavor; and 2.23 g or 6.97% by weight of purified water.

In some embodiments of the invention, the product may be for providing relief of minor aches and pains and/or temporary water-weight gain, bloating, swelling, and/or full feeling associated with the premenstrual and menstrual periods.

In some embodiments of the invention, the gel is for treating headache. The gel for treating headache may include acetaminophen, pectin, honey, glycerin, ascorbic acid, citric acid, strawberry flavor, and water. The pectin is preferably slow set high methoxyl pectin. The honey is preferably Manuka honey. The strawberry flavor is preferably natural strawberry flavor. The water is preferably purified water. The gel storage and delivery device preferably contains 32 grams of the gel. The gel preferably contains 220-600 mg or 0.687-1.875% by weight of acetaminophen; 60-260 mg or 0.18-0.81% by weight of pectin; 16,000-30,400 mg or 50-95% by weight of honey; 160-640 mg or 0.5-2% by weight of glycerin; 16-128 mg or 0.05-0.4% by weight of ascorbic acid; 16-128 mg or 0.05-0.4% by weight of citric acid; 32-320 mg or 0.1-1.0% by weight raspberry or strawberry flavor; and 960-3200 mg or 3-10% by weight of water. In one preferred embodiment, the gel for treating headache comprises 0.5 g or 1.56% by weight of acetaminophen; 0.16 g or 0.50% by weight of slow set high methoxyl pectin; 28.43 g or 88.84% by weight of Manuka honey; 0.48 g or 1.50% by weight of glycerin; 0.04 g or 0.13% by weight of ascorbic acid; 0.06 g or 0.19% by weight of citric acid; 0.1 g or 0.31% by weight of natural strawberry flavor; and 2.23 g or 6.97% by weight of purified water. Alternatively, any desired flavor such as a natural fruit flavor or flavors may be used.

In other embodiments of the invention, the gel for treating headache may include other active pharmaceutical ingredients such as ibuprofen, aspirin, naproxen, diclofenac, ketorolac, celecoxib and/or excedrin (combination of three APIs: acetaminophen, aspirin, caffeine).

The gel for treating headache may also include herbal ingredients such as feverfew, butterbur, peppermint, ginger, willow bark and/or lavender.

Optionally, the gel may incorporate a pH buffer such as sodium citrate. For example, the gel for treating headache may include acetaminophen, pectin, honey, glycerin, ascorbic acid, citric acid, sodium citrate, strawberry flavor, and water. The pectin is preferably slow set high methoxyl pectin. The honey is preferably Manuka honey. The strawberry flavor is preferably natural strawberry flavor. The water is preferably purified water. The gel storage and delivery device preferably contains 32 grams of the gel. The gel preferably contains 220-600 mg or 0.687-1.875% by weight of acetaminophen; 60-260 mg or 0.18-0.81% by weight of pectin; 16,000-30,400 mg or 50-95% by weight of honey; 160-640 mg or 0.5-2% by weight of glycerin; 16-128 mg or 0.05-0.4% by weight of ascorbic acid; 16-128 mg or 0.05-0.4% by weight of citric acid; 8-64 mg or 0.025-0.2% by weight of sodium citrate; 32-320 mg or 0.1-1.0% by weight raspberry or strawberry flavor; and 960-3200 mg or 3-10% by weight of water. In one preferred embodiment, the gel for treating headache comprises 0.5 g or 1.56% by weight of acetaminophen; 0.16 g or 0.50% by weight of slow set high methoxyl pectin; 28.37 g or 88.66% by weight of Manuka honey; 0.48 g or 1.50% by weight of glycerin; 0.04 g or 0.13% by weight of ascorbic acid; 0.06 g or 0.19% by weight of citric acid; 0.06 g or 0.19% by weight of sodium citrate; 0.1 g or 0.31% by weight of natural strawberry flavor; and 2.23 g or 6.97% by weight of purified water.

In some embodiments of the invention, the product is for relieving minor aches and pains, for example due to headache, arthritis, backache, and/or common cold, and/or for reducing fever.

In other embodiments of the invention, the gel may be for treating nausea and/or upset stomach. The gel for treating nausea and/or upset stomach may incorporate active pharmaceutical ingredients such as bismuth subsalicylate, dimenhydrinate, meclizine hydrochloride, phosphorated carbohydrate, sodium citrate dehydrate, doxylamine succinate, and/or pyridoxine hydrochloride. The bismuth subsalicylate may, for example, be included in a dosage of 524 mg. The dimenhydrinate may, for example, be included in a dosage of 50–100 mg. The meclizine hydrochloride may, for example, be included in a dosage of 25–50 mg. The phosphorated carbohydrate (dextrose/fructose/phosphoric acid) may, for example, be included in a dosage of 15–30 mL. The sodium citrate dehydrate may, for example, be included in a dosage of 2 to 4 g. The doxylamine succinate may, for example, be included in a dosage of 12.5–25 mg. The pyridoxine hydrochloride may, for example, be included in a dosage of 10–25 mg. Excipients such as ginger, peppermint, chamomile, fennel, lemon balm, and/or licorice root could also be included in the gel for treating nausea and/or upset stomach.

Although several embodiments of the invention described herein provide a product in the form of a gel, this is not necessary. Rather, the invention could be used with any suitable fluid, wherein the term “fluid” is intended to refer broadly to any flowable substance such as liquids, liquid solutions, oral suspensions, liquid semi-solid oral doses, creams, gels, foams, dispersions, pastes, Newtonian fluids, non-Newtonian fluids, emulsions, suspensions, viscoelastic materials, viscoelastic solutions, viscoelastic fluids, viscoelastic food gels, and/or colloids. The invention is particularly advantageous for delivering oral dosages of fluids that are relatively viscous, and which thus cannot be easily consumed by pouring the fluids out of a cup or container. In some preferred embodiments of the invention, the fluid has a viscosity of at least 300 mPa·s (all viscosity values are measured at 25 degrees Celsius, unless otherwise noted). In some embodiments, the viscosity of the fluid is between 300 mPa·s and 2000 mPa·s. In some embodiments, the viscosity is at least 600 mPa·s. In some embodiments, the viscosity is between 600 mPa·s and 1000 mPa·s. In some embodiments, the fluid has a viscosity similar to that of syrup and/or honey.

Although this disclosure has described and illustrated certain preferred embodiments of the invention, it is to be understood that the invention is not restricted to these particular embodiments. Rather, the invention includes all embodiments which are functional, chemical, medical and/or mechanical equivalents of the specific embodiments and features that have been described and illustrated herein.

### Items

Item 1.: A sleep aid comprising a gel storage and delivery device.

Item 2.: The sleep aid of any other item comprising a gel.

Item 3.: The sleep aid of any other item wherein the gel comprises pectin.

Item 4.: The sleep aid of any other item wherein the gel comprises diphenhydramine.

Item 5.: The sleep aid of any other item wherein the gel storage and delivery device comprises a collapsible chamber that contains the gel.

Item 6.: The sleep aid of any other item wherein the gel storage and delivery device comprises a delivery portion for delivering the gel from the collapsible chamber to a user’s mouth.

Item 7.: The sleep aid of any other item

wherein the collapsible chamber collapses from an initial state having a first internal volume to a collapsed state having a second internal volume as the gel is delivered from the collapsible chamber to the user’s mouth, the first internal volume being greater than the second internal volume.

Item 8.: The sleep aid of any other item, wherein the gel storage and delivery device is configured to deliver the gel to the user’s mouth on application of a delivery force that creates a pressure differential between the collapsible chamber and the user’s mouth.

Item 9.: The sleep aid of any other item, wherein the delivery force comprises at least one of:

a vacuum force applied by the user’s mouth to the delivery portion; and

a compressive force applied manually to the collapsible chamber.

Item 10.: The sleep aid of any other item, wherein the gel storage and delivery device comprises a flexible pouch.

Item 11.: The sleep aid of any other item, wherein the flexible pouch has a base portion that is configured to engage with a horizontal support surface to support the flexible pouch in an upright orientation when the collapsible chamber is in the initial state.

Item 12.: The sleep aid of any other item, wherein the delivery portion is at an uppermost end of the flexible pouch when in the upright orientation.

Item 13.: The sleep aid of any other item, wherein the delivery portion comprises a severable section of the flexible pouch;

wherein, when the severable section is intact, the delivery portion is sealed and the gel is prevented from exiting the flexible pouch; and

wherein, when the severable section is ruptured, the delivery portion is open and the gel is able to be delivered from the flexible pouch.

Item 14.: The sleep aid of any other item, wherein the severable section is configured to be manually tearable.

Item 15.: The sleep aid of any other item, wherein the flexible pouch has a main pouch body with a pouch body width; and

wherein the severable section has a narrower width than the main pouch body.

Item 16.: The sleep aid of any other item, wherein the pectin comprises methoxyl pectin.

Item 17.: The sleep aid of any other item, wherein the pectin comprises high methoxyl pectin.

Item 18.: The sleep aid of any other item, wherein the pectin comprises high methoxyl slow set pectin.

Item 19.: The sleep aid of any other item, wherein the diphenhydramine comprises diphenhydramine hydrochloride.

Item 20.: The sleep aid of any other item, wherein the gel storage and delivery device contains only one dose of the gel.

Item 21.: The sleep aid of any other item, wherein the gel storage and delivery device contains no more than 50 grams of the gel.

Item 22.: The sleep aid of any other item, wherein the gel storage and delivery device contains at least 10 grams of the gel.

Item 23.: The sleep aid of any other item, wherein the gel storage and delivery device contains between 25 grams and 40 grams of the gel.

Item 24.: The sleep aid of any other item, wherein the gel storage and delivery device contains about 32 grams of the gel.

Item 25.: The sleep aid of any other item, wherein the gel storage and delivery device contains no more than 100 mg of diphenhydramine.

Item 26.: The sleep aid of any other item, wherein the gel storage and delivery device contains at least 5 mg of diphenhydramine.

Item 27.: The sleep aid of any other item, wherein the gel storage and delivery device contains from 25 mg to 50 mg of diphenhydramine.

Item 28.: The sleep aid of any other item, wherein the gel storage and delivery device contains about 25 mg of diphenhydramine.

Item 29.: The sleep aid of any other item, wherein the gel storage and delivery device contains about 50 mg of diphenhydramine.

Item 30.: The sleep aid of any other item, wherein the gel storage and delivery device contains about 48 mg of diphenhydramine.

Item 31.: The sleep aid of any other item, wherein the gel contains no more than 1.5% pectin by weight.

Item 32.: The sleep aid of any other item, wherein the gel contains at least 0.25% pectin by weight.

Item 33.: The sleep aid of any other item, wherein the gel contains about 0.8% pectin by weight.

Item 34.: The sleep aid of any other item, wherein the gel contains about 0.5% pectin by weight.

Item 35.: The sleep aid of any other item, wherein the gel contains honey.

Item 36.: The sleep aid of any other item, wherein the gel contains at least 75% honey by weight.

Item 37.: The sleep aid of any other item, wherein the gel contains no more than 95% honey by weight.

Item 38.: The sleep aid of any other item, wherein the gel contains 85% to 90% honey by weight.

Item 39.: The sleep aid of any other item, wherein the gel contains tart cherry.

Item 40.: The sleep aid of any other item, wherein the gel contains about 0.5% tart cherry by weight.

Item 41.: The sleep aid of any other item, wherein the gel contains water.

Item 42.: The sleep aid of any other item, wherein the gel contains about 7.5% water by weight.

Item 43.: The sleep aid of any other item, wherein the gel contains glycerin.

Item 44.: The sleep aid of any other item, wherein the gel contains about 1.5% glycerin by weight.

Item 45.: The sleep aid of any other item, wherein the gel contains ascorbic acid.

Item 46.: The sleep aid of any other item, wherein the gel contains about 0.1% ascorbic acid by weight.

Item 47.: The sleep aid of any other item, wherein the gel contains citric acid.

Item 48.: The sleep aid of any other item, wherein the gel contains about 0.1% citric acid by weight.

Item 49.: The sleep aid of any other item, wherein the gel contains potassium sorbate.

Item 50.: The sleep aid of any other item, wherein the gel contains about 0.05% potassium sorbate by weight.

Item 51.: The sleep aid of any other item, wherein the gel contains cherry flavor.

Item 52.: The sleep aid of any other item, wherein the gel contains about 0.5% cherry flavor by weight.

Item 53.: The sleep aid of any other item, wherein the gel contains Piper longum.

Item 54.: The sleep aid of any other item, wherein the gel contains about 1% Piper longum by weight.

Item 55.: The sleep aid of any other item, wherein the gel contains Valeriana wallichii.

Item 56.: The sleep aid of any other item, wherein the gel contains about 0.5% Valeriana wallichii by weight.

Item 57.: The sleep aid of any other item, wherein the gel contains Melissa officinalis.

Item 58.: The sleep aid of any other item, wherein the gel contains about 0.75% Melissa officinalis by weight.

Item 59.: The sleep aid of any other item, wherein the gel contains Rosmarinus officinalis.

Item 60.: The sleep aid of any other item, wherein the gel contains about 0.75% Rosmarinus officinalis by weight.

Item 61.: The sleep aid of any other item, wherein the gel contains:

about 0.15% diphenhydramine by weight;

0.25% to 1.5% pectin by weight;

0.25% to 1.25% tart cherry by weight;

75% to 95% honey by weight;

0.5% to 2.5 % glycerin by weight;

0.05% to 0.5% ascorbic acid by weight;

0.05% to 0.5% citric acid by weight;

0.05% to 0.5% potassium sorbate by weight;

0.1% to 1% cherry flavor by weight;

0.25% to 1.5% Piper longum by weight;

0.25% to 1.5% Valeriana wallichii by weight;

0.25% to 1.5% Melissa officinalis by weight;

0.25% to 1.5% Rosmarinus officinalis by weight; and

5% to 15% water by weight.

Item 62.: The sleep aid of any other item, wherein the gel contains:

0.15% diphenhydramine by weight;

0.8% high methoxyl slow set pectin by weight;

0.5% tart cherry by weight;

85.8% honey by weight;

1.5% glycerin by weight;

0.1% ascorbic acid by weight;

0.1% citric acid by weight;

0.05% potassium sorbate by weight;

0.5% cherry flavor by weight;

1% Piper longum by weight;

0.5% Valeriana wallichii by weight;

0.75% Melissa officinalis by weight;

0.75% Rosmarinus officinalis by weight; and

7.5% water by weight.

Item 63.: The sleep aid of any other item, wherein the gel contains:

0.15% diphenhydramine by weight;

0.5% high methoxyl slow set pectin by weight;

0.5% tart cherry by weight;

87.35% honey by weight;

1.5% glycerin by weight;

0.1% ascorbic acid by weight;

0.1% citric acid by weight;

0.05% potassium sorbate by weight;

0.5% cherry flavor by weight;

0.5% Piper longum by weight;

0.25% Valeriana wallichii by weight;

0.5% Melissa officinalis by weight;

0.5% Rosmarinus officinalis by weight; and

7.5% water by weight.

Item 64.: Use of the sleep aid of any other item to promote sleep.

Item 65.: Use of the sleep aid of any other item to treat insomnia.

Item 66.: A method comprising:

providing the sleep aid of any other item; and

instructing a user to ingest the gel in order to promote sleep.

Item 67.: A method comprising:

providing the sleep aid of any other item; and

instructing a user to ingest the gel in order to treat insomnia.

Item 68.: A method comprising:

obtaining the sleep aid of any other item; and

ingesting the gel in order to promote sleep.

Item 69.: A method comprising:

obtaining the sleep aid of any other item; and

ingesting the gel in order to treat insomnia.

Item 70.: A sleep aid comprising:

a gel storage and delivery device; and

a gel containing a sleep promoting ingredient.

Item 71.: The sleep aid of any other item wherein the gel storage and delivery device comprises:

a collapsible chamber that contains the gel; and

a delivery portion for delivering the gel from the collapsible chamber to a user’s mouth;

wherein the collapsible chamber collapses from an initial state having a first internal volume to a collapsed state having a second internal volume as the gel is delivered from the collapsible chamber to the user’s mouth, the first internal volume being greater than the second internal volume.

Item 72.: The sleep aid of any other item, wherein the sleep promoting ingredient comprises diphenhydramine.

Item 73.: The sleep aid of any other item, wherein the gel contains pectin.

Item 74.: A product comprising:

a gel storage and delivery device; and

a gel.

Item 75.: The product of any other item wherein the gel comprises pectin.

Item 76.: The product of any other item wherein the gel comprises diphenhydramine.

Item 77.: The product of any other item wherein the gel storage and delivery device comprises a collapsible chamber that contains the gel.

Item 78.: The product of any other item wherein the gel storage and delivery device comprises a delivery portion for delivering the gel from the collapsible chamber to a user’s mouth;

wherein the collapsible chamber collapses from an initial state having a first internal volume to a collapsed state having a second internal volume as the gel is delivered from the collapsible chamber to the user’s mouth, the first internal volume being greater than the second internal volume.

Item 79.: A pharmaceutical product comprising:

a gel storage and delivery device; and

a gel containing a synthetic active pharmaceutical ingredient.

Item 80.: The pharmaceutical product of any other item wherein the gel storage and delivery device comprises:

a collapsible chamber that contains the gel; and

a delivery portion for delivering the gel from the collapsible chamber to a user’s mouth;

wherein the collapsible chamber collapses from an initial state having a first internal volume to a collapsed state having a second internal volume as the gel is delivered from the collapsible chamber to the user’s mouth, the first internal volume being greater than the second internal volume.

Item 81.: The pharmaceutical product of any other item, wherein the gel further comprises a bioavailability enhancer that increases the bioavailability of the synthetic active pharmaceutical ingredient.

Item 82.: The pharmaceutical product of any other item, wherein the bioavailability enhancer comprises a gelling agent.

Item 83.: The pharmaceutical product of any other item, wherein the bioavailability enhancer comprises a polymer.

Item 84.: The pharmaceutical product of any other item, wherein the bioavailability enhancer comprises at least one of: pectin, acacia, agar, methylcellulose, tragacanth, gelatin, collagen, guar gum, alginate, cornstarch, and chia seeds.

Item 85.: The pharmaceutical product of any other item, wherein the bioavailability enhancer comprises pectin.

Item 86.: The pharmaceutical product of any other item, wherein the pharmaceutical product is a sleep aid.

Item 87.: The pharmaceutical product of any other item, wherein the synthetic active pharmaceutical ingredient comprises a drug that promotes sleep.

Item 88.: The pharmaceutical product of any other item, wherein the synthetic active pharmaceutical ingredient comprises at least one of: diphenhydramine, cetirizine, trazodone, zolpidem, and fexofenadine.

Item 89.: The pharmaceutical product of any other item, wherein the synthetic active pharmaceutical ingredient comprises diphenhydramine.

Item 90.: The pharmaceutical product of any other item, wherein the gel storage and delivery device comprises a flexible pouch.

Item 91.: The pharmaceutical product of any other item, wherein the gel storage and delivery device comprises a squeeze pack.

Item 92.: The pharmaceutical product of any other item, wherein the synthetic active pharmaceutical ingredient comprises at least one of: an anti-inflammatory, a pain reliever, an antihistamine, a decongestant, a cough suppressant, an expectorant, a weight loss medication, an antidiarrheal, a laxative, an anti-nausea medication, a motion sickness medication, an antidepressant, an antipsychotic, an anti-anxiety medication, an addiction treatment medication, an antiviral medication, an antibiotic, a statin, an antihypertensive medication, a synthetic hormone, an antidiabetic, a stomach acid reduction medication, a contraceptive, a diuretic, a bronchodilator, an anticonvulsant, a synthetic steroid, an anticoagulant, a synthetic stimulant, a non-steroidal anti-inflammatory drug, an analgesic, and an antipyretic.

Item 93.: The pharmaceutical product of any other item, wherein the synthetic active pharmaceutical ingredient comprises ibuprofen.

Item 94.: The pharmaceutical product of any other item, wherein the synthetic active pharmaceutical ingredient comprises acetaminophen.

Item 95.: A pharmaceutical product comprising:

a storage and delivery device; and

a fluid containing a synthetic active pharmaceutical ingredient.

Item 96.: The pharmaceutical product of any other item wherein the storage and delivery device comprises:

a collapsible chamber that contains the fluid; and

a delivery portion for delivering the fluid from the collapsible chamber to a user’s mouth;

wherein the collapsible chamber collapses from an initial state having a first internal volume to a collapsed state having a second internal volume as the fluid is delivered from the collapsible chamber to the user’s mouth, the first internal volume being greater than the second internal volume.

Item 97.: The pharmaceutical product of any other item, wherein the fluid further comprises a bioavailability enhancer that increases the bioavailability of the synthetic active pharmaceutical ingredient.

Item 98.: The pharmaceutical product of any other item, wherein the bioavailability enhancer comprises a viscosity modifying agent that increases the viscosity of the fluid.

Item 99.: The pharmaceutical product of any other item, wherein the fluid further comprises a viscosity modifying agent that increases the viscosity of the fluid.

Item 100.: The pharmaceutical product of any other item, wherein the fluid comprises at least one of: a liquid, a dispersion, a foam, a paste, a Newtonian fluid, a non-Newtonian fluid, an emulsion, a suspension, and a colloid.

Item 101.: The pharmaceutical product of any other item, wherein the fluid has a viscosity greater than 1500 mPA·s.

Item 102.: The pharmaceutical product of any other item, wherein the fluid has a viscosity lower than 2500 mPA·s.

Item 103.: The pharmaceutical product of any other item, wherein the fluid has a viscosity from 1900 mPA·s to 2500 mPA·s.

Item 104.: The pharmaceutical product of any other item, wherein the gel has a viscosity greater than 1500 mPA·s.

Item 105.: The pharmaceutical product of any other item and 91, wherein the gel has a viscosity lower than 2500 mPA·s.

Item 106.: The pharmaceutical product of any other item, wherein the gel has a viscosity from 1900 mPA·s to 2500 mPA·s.

Item 107.: A pharmaceutical product comprising:

a storage and delivery device; and

a fluid containing an active pharmaceutical ingredient in an oral dosage form.

Item 108.: The pharmaceutical product of any other item wherein the storage and delivery device comprises:

a collapsible chamber that contains the fluid; and

a delivery portion for delivering the fluid from the collapsible chamber to a user’s mouth for ingestion;

wherein the collapsible chamber collapses from an initial state having a first internal volume to a collapsed state having a second internal volume as the fluid is delivered from the collapsible chamber to the user’s mouth, the first internal volume being greater than the second internal volume.

Item 109.: The pharmaceutical product of any other item, wherein the active pharmaceutical ingredient comprises a synthetic active pharmaceutical ingredient.

Item 110.: The pharmaceutical product of any other item, wherein the fluid comprises a bioavailability enhancer that increases the bioavailability of the active pharmaceutical ingredient.

Item 111.: The pharmaceutical product of any other item, wherein the bioavailability enhancer comprises a gelling agent.

Item 112.: The pharmaceutical product of any other item, wherein the bioavailability enhancer comprises a polymer.

Item 113.: The pharmaceutical product of any other item, wherein the bioavailability enhancer comprises at least one of: pectin, acacia, agar, methylcellulose, tragacanth, gelatin, collagen, guar gum, alginate, cornstarch, and chia seeds.

Item 114.: The pharmaceutical product of any other item, wherein the fluid comprises pectin.

Item 115.: The pharmaceutical product of any other item, wherein the storage and delivery device comprises a flexible pouch.

Item 116.: The pharmaceutical product of any other item, wherein the storage and delivery device comprises a squeeze pack.

Item 117.: The pharmaceutical product of any other item, wherein the pharmaceutical product is a sleep aid.

Item 118.: The pharmaceutical product of any other item, wherein the active pharmaceutical ingredient comprises a drug that promotes sleep.

Item 119.: The pharmaceutical product of any other item, wherein active pharmaceutical ingredient comprises at least one of: diphenhydramine, cetirizine, trazodone, zolpidem, fexofenadine, doxylamine succinate, and promethazine.

Item 120.: The pharmaceutical product of any other item, wherein the active pharmaceutical ingredient comprises diphenhydramine.

Item 121.: The pharmaceutical product of any other item, wherein the fluid comprises tart cherry.

Item 122.: The pharmaceutical product of any other item, wherein the fluid comprises honey.

Item 123.: The pharmaceutical product of any other item, wherein the fluid comprises glycerin.

Item 124.: The pharmaceutical product of any other item, wherein the fluid comprises ascorbic acid.

Item 125.: The pharmaceutical product of any other item, wherein the fluid comprises citric acid.

Item 126.: The pharmaceutical product of any other item, wherein the fluid comprises cherry flavor.

Item 127.: The pharmaceutical product of any other item, wherein the fluid comprises water.

Item 128.: The pharmaceutical product of any other item, wherein the storage and delivery device contains about 32 grams of the fluid.

Item 129.: The pharmaceutical product of any other item, wherein the fluid contains 15 to 50 mg of diphenhydramine.

Item 130.: The pharmaceutical product of any other item, wherein the fluid contains 0.046 to 0.16% by weight of diphenhydramine.

Item 131.: The pharmaceutical product of any other item, wherein the fluid contains 60 to 260 mg of pectin.

Item 132.: The pharmaceutical product of any other item, wherein the fluid contains 0.18 to 0.81% by weight of pectin.

Item 133.: The pharmaceutical product of any other item, wherein the fluid contains 80 to 200 mg of tart cherry.

Item 134.: The pharmaceutical product of any other item, wherein the fluid contains 0.25 to 0.625% by weight of tart cherry.

Item 135.: The pharmaceutical product of any other item, wherein the fluid contains 16,000 to 30,400 mg of honey.

Item 136.: The pharmaceutical product of any other item, wherein the fluid contains 50 to 95% by weight of honey.

Item 137.: The pharmaceutical product of any other item, wherein the fluid contains 160 to 640 mg of glycerin.

Item 138.: The pharmaceutical product of any other item, wherein the fluid contains 0.5 to 2% by weight of glycerin.

Item 139.: The pharmaceutical product of any other item, wherein the fluid contains 16 to 128 mg of ascorbic acid.

Item 140.: The pharmaceutical product of any other item, wherein the fluid contains 0.05 to 0.4% by weight of ascorbic acid.

Item 141.: The pharmaceutical product of any other item, wherein the fluid contains 16 to 128 mg of citric acid.

Item 142.: The pharmaceutical product of any other item, wherein the fluid contains 0.05 to 0.4% by weight of citric acid.

Item 143.: The pharmaceutical product of any other item, wherein the fluid contains 32 to 320 mg of cherry flavor.

Item 144.: The pharmaceutical product of any other item, wherein the fluid contains 0.1 to 1.0% by weight of cherry flavor.

Item 145.: The pharmaceutical product of any other item, wherein the fluid contains 960 to 3200 mg of water.

Item 146.: The pharmaceutical product of any other item, wherein the fluid contains 3 to 10% by weight of water.

Item 147.: The pharmaceutical product of any other item, wherein the fluid contains:

about 0.05 g or about 0.16% by weight of diphenhydramine hydrochloride;

about 0.16 g or about 0.50% by weight of slow set high methoxyl pectin;

about 0.16 g or about 0.50% by weight of tart cherry (Prunus Cerasus);

about 28.5260 g or about 89.14% by weight of Manuka honey;

about 0.48 g or about 1.50% by weight of glycerin;

about 0.032 g or about 0.10% by weight of ascorbic acid;

about 0.032 g or about 0.10% by weight of citric acid;

about 0.16 g or about 0.50% by weight of natural cherry flavor; and

about 2.4 g or about 7.50% by weight of purified water.

Item 148.: The pharmaceutical product of any other item, wherein the fluid contains sodium citrate.

Item 149.: The pharmaceutical product of any other item, wherein the fluid contains 8 to 64 mg of sodium citrate.

Item 150.: The pharmaceutical product of any other item, wherein the fluid contains 0.025 to 0.2% by weight of sodium citrate.

Item 151.: The pharmaceutical product of any other item, wherein the fluid contains:

about 0.05 g or about 0.16% by weight of diphenhydramine hydrochloride;

about 0.16 g or about 0.50% by weight of slow set high methoxyl pectin;

about 0.16 g or about 0.50% by weight of tart cherry (Prunus Cerasus);

about 28.494 g or about 89.04% by weight of Manuka honey;

about 0.48 g or about 1.50% by weight of glycerin;

about 0.032 g or about 0.10% by weight of ascorbic acid;

about 0.032 g or about 0.10% by weight of citric acid;

about 0.032 g or about 0.10% by weight of sodium citrate;

about 0.16 g or about 0.50% by weight of natural cherry flavor; and

about 2.4 g or about 7.50% by weight of purified water.

Item 152.: The pharmaceutical product of any other item, wherein the fluid is for treating pain.

Item 153.: The pharmaceutical product of any other item, wherein the fluid is for treating menstrual pain.

Item 154.: The pharmaceutical product of any other item, wherein the fluid is for treating headache.

Item 155.: The pharmaceutical product of any other item, wherein the fluid comprises at least one of: acetaminophen, ibuprofen, naproxen, aspirin, celecoxib, diclofenac, ketoprofen, mefenamic acid, magnesium salicylate, ketorolac, and excedrin.

Item 156.: The pharmaceutical product of any other item, wherein the fluid comprises acetaminophen.

Item 157.: The pharmaceutical product of any other item, wherein the fluid comprises pamabrom.

Item 158.: The pharmaceutical product of any other item, wherein the fluid comprises honey.

Item 159.: The pharmaceutical product of any other item, wherein the fluid comprises glycerin.

Item 160.: The pharmaceutical product of any other item, wherein the fluid comprises ascorbic acid.

Item 161.: The pharmaceutical product of any other item, wherein the fluid comprises citric acid.

Item 162.: The pharmaceutical product of any other item, wherein the fluid comprises strawberry flavor.

Item 163.: The pharmaceutical product of any other item, wherein the fluid comprises water.

Item 164.: The pharmaceutical product of any other item, wherein the storage and delivery device contains about 32 grams of the fluid.

Item 165.: The pharmaceutical product of any other item, wherein the fluid contains 220 to 600 mg of acetaminophen.

Item 166.: The pharmaceutical product of any other item, wherein the fluid contains 0.687 to 1.875% by weight of acetaminophen.

Item 167.: The pharmaceutical product of any other item, wherein the fluid contains 5 to 25 mg of pamabrom.

Item 168.: The pharmaceutical product of any other item, wherein the fluid contains 0.0156 to 0.078% by weight of pamabrom.

Item 169.: The pharmaceutical product of any other item, wherein the fluid contains 60 to 260 mg of pectin.

Item 170.: The pharmaceutical product of any other item, wherein the fluid contains 0.18 to 0.81% by weight of pectin.

Item 171.: The pharmaceutical product of any other item, wherein the fluid contains 16,000 to 30,400 mg of honey.

Item 172.: The pharmaceutical product of any other item, wherein the fluid contains 50 to 95% by weight of honey.

Item 173.: The pharmaceutical product of any other item, wherein the fluid contains 160 to 640 mg of glycerin.

Item 174.: The pharmaceutical product of any other item, wherein the fluid contains 0.5 to 2% by weight of glycerin.

Item 175.: The pharmaceutical product of any other item, wherein the fluid contains 16 to 128 mg of ascorbic acid.

Item 176.: The pharmaceutical product of any other item, wherein the fluid contains 0.05 to 0.4% by weight of ascorbic acid.

Item 177.: The pharmaceutical product of any other item, wherein the fluid contains 16 to 128 mg of citric acid.

Item 178.: The pharmaceutical product of any other item, wherein the fluid contains 0.05 to 0.4% by weight of citric acid.

Item 179.: The pharmaceutical product of any other item, wherein the fluid contains 32 to 320 mg of strawberry flavor.

Item 180.: The pharmaceutical product of any other item, wherein the fluid contains 0.1 to 1.0% by weight of strawberry flavor.

Item 181.: The pharmaceutical product of any other item, wherein the fluid contains 960 to 3200 mg of water.

Item 182.: The pharmaceutical product of any other item, wherein the fluid contains 3 to 10% by weight of water.

Item 183.: The pharmaceutical product of any other item, wherein the fluid contains:

about 0.5 g or about 1.56% by weight of acetaminophen;

about 0.025 g or about 0.08% by weight of pamabrom;

about 0.16 g or about 0.50% by weight of slow set high methoxyl pectin;

about 28.405 g or about 88.77% by weight of Manuka honey;

about 0.48 g or about 1.50% by weight of glycerin;

about 0.04 g or about 0.13% by weight of ascorbic acid;

about 0.06 g or about 0.19% by weight of citric acid;

about 0.1 g or about 0.31% by weight of natural strawberry flavor; and

about 2.23 g or about 6.97% by weight of purified water.

Item 184.: The pharmaceutical product of any other item, wherein the fluid comprises sodium citrate.

Item 185.: The pharmaceutical product of any other item, wherein the fluid contains 8 to 64 mg of sodium citrate.

Item 186.: The pharmaceutical product of any other item, wherein the fluid contains 0.025 to 0.2% by weight of sodium citrate.

Item 187.: The pharmaceutical product of any other item, wherein the fluid contains:

about 0.5 g or about 1.56% by weight of acetaminophen;

about 0.025 g or about 0.08% by weight of pamabrom;

about 0.16 g or about 0.50% by weight of slow set high methoxyl pectin;

about 28.345 g or about 88.58% by weight of Manuka honey;

about 0.48 g or about 1.50% by weight of glycerin;

about 0.04 g or about 0.13% by weight of ascorbic acid;

about 0.06 g or about 0.19% by weight of citric acid;

about 0.06 g or about 0.19% by weight of sodium citrate;

about 0.1 g or about 0.31% by weight of natural strawberry flavor; and

about 2.23 g or about 6.97% by weight of purified water.

Item 188.: The pharmaceutical product of any other item, wherein the fluid contains:

about 0.5 g or about 1.56% by weight of acetaminophen;

about 0.16 g or about 0.50% by weight of slow set high methoxyl pectin;

about 28.43 g or about 88.84% by weight of Manuka honey;

about 0.48 g or about 1.50% by weight of glycerin;

about 0.04 g or about 0.13% by weight of ascorbic acid;

about 0.06 g or about 0.19% by weight of citric acid;

about 0.1 g or about 0.31% by weight of natural strawberry flavor; and

about 2.23 g or about 6.97% by weight of purified water.

Item 189.: The pharmaceutical product of any other item, wherein the fluid contains:

about 0.5 g or about 1.56% by weight of acetaminophen;

about 0.16 g or about 0.50% by weight of slow set high methoxyl pectin;

about 28.43 g or about 88.84% by weight of Manuka honey;

about 0.48 g or about 1.50% by weight of glycerin;

about 0.04 g or about 0.13% by weight of ascorbic acid;

about 0.06 g or about 0.19% by weight of citric acid;

about 0.1 g or about 0.31% by weight of natural strawberry flavor; and

about 2.23 g or about 6.97% by weight of purified water.

Item 190.: The pharmaceutical product of any other item, wherein the fluid contains:

about 0.5 g or about 1.56% by weight of acetaminophen;

about 0.16 g or about 0.50% by weight of slow set high methoxyl pectin;

about 28.37 g or about 88.66% by weight of Manuka honey;

about 0.48 g or about 1.50% by weight of glycerin;

about 0.04 g or about 0.13% by weight of ascorbic acid;

about 0.06 g or about 0.19% by weight of citric acid;

about 0.06 g or about 0.19% by weight of sodium citrate;

about 0.1 g or about 0.31% by weight of natural strawberry flavor; and

about 2.23 g or about 6.97% by weight of purified water.

Item 191.: The pharmaceutical product of any other item, wherein the fluid is for treating nausea.

Item 192.: The pharmaceutical product of any other item, wherein the fluid is for treating upset stomach.

Item 193.: The pharmaceutical product of any other item, wherein the fluid comprises at least one of: bismuth subsalicylate, dimenhydrinate, meclizine hydrochloride, phosphorated carbohydrate, sodium citrate dehydrate, doxylamine succinate, and pyridoxine hydrochloride.

Item 194.: The pharmaceutical product of any other item, wherein the fluid has a viscosity of at least 300 mPa·s at 25 degrees Celsius.

Item 195.: The pharmaceutical product of any other item, wherein the fluid has a viscosity between 300 mPa·s and 2000 mPa·s at 25 degrees Celsius.

Item 196.: The pharmaceutical product of any other item, wherein the fluid has a viscosity of at least 600 mPa·s at 25 degrees Celsius.

Item 197.: The pharmaceutical product of any other item, wherein the fluid has a viscosity between 600 mPa·s and 1000 mPa·s at 25 degrees Celsius.

Item 198.: The pharmaceutical product of any other item, wherein the fluid comprises a gel.

Item 199.: A sleep aid comprising:

a storage and delivery device; and

a fluid.

Item 200.: The sleep aid of any other item wherein the fluid comprises pectin.

Item 201.: The sleep aid of any other item wherein the fluid comprises diphenhydramine in an oral dosage form.

Item 202.: The sleep aid of any other item wherein the storage and delivery device comprises:

a collapsible chamber that contains the fluid; and

a delivery portion for delivering the fluid from the collapsible chamber to a user’s mouth for ingestion;

wherein the collapsible chamber collapses from an initial state having a first internal volume to a collapsed state having a second internal volume as the fluid is delivered from the collapsible chamber to the user’s mouth, the first internal volume being greater than the second internal volume.

Item 203.: The sleep aid of any other item, wherein the fluid has a viscosity of at least 300 mPa·s at 25 degrees Celsius.

Item 204.: The sleep aid of any other item, wherein the fluid comprises a gel.

We claim:

1. A single-use sleep aid comprising:
a disposable gel storage and delivery device comprising a collapsible chamber and a delivery portion, the delivery portion for delivering the gel from the collapsible chamber to a user’s mouth, the delivery portion having a severable section that is manually severable to permanently unseal the delivery portion; and
a single dose of 10 grams to 50 grams of a gel having a viscosity of between 600 mPa·s and 1000 mPa·s at 25 degrees Celsius, the gel located within the collapsible chamber and the gel comprising:
0.046% to 0.32% by weight diphenhydramine hydrochloride sleep aid,
0.25% to 1.5% by weight of high methoxyl slow set pectin with a degree of methylation greater than 50%, the high methoxyl slow set pectin being a bioavailability enhancer and slow release agent for diphenhydramine,
0.25% to 1.25% by weight Prunus Cerasus containing melatonin precursor,
0.05% to 0.5% by weight citric acid pectin activator,
75% to 95% by weight honey,
0.5% to 2.5% by weight glycerin preservative, and
0.05% to 0.5% by weight ascorbic acid antioxidant and preservative;
wherein the collapsible chamber is collapsible from an initial state having a first internal volume to a collapsed state having a second internal volume as the gel is delivered from the collapsible chamber to the user’s mouth through the delivery portion, the first internal volume being greater than the second internal volume.

2. The sleep aid according to claim 1, wherein the gel storage and delivery device is configured to deliver the gel to the user’s mouth on application of a delivery force that creates a pressure differential between the collapsible chamber and the user’s mouth.

3. The sleep aid according to claim 1, wherein the gel storage and delivery device comprises a flexible pouch.

4. The sleep aid according to claim 1 wherein, when the severable section is intact, the delivery portion is sealed and the gel is prevented from exiting the flexible pouch.

5. The sleep aid according to claim 4, wherein the severable section is configured to be manually tearable.

6. The sleep aid according to claim 1, wherein the gel storage and delivery device contains no more than 50 mg of diphenhydramine hydrochloride sleep aid.

7. The sleep aid according to claim 1, wherein the gel storage and delivery device contains about 50 mg of diphenhydramine hydrochloride sleep aid.

8. The sleep aid according to claim 1, wherein the gel contains about 0.5% by weight of the high methoxyl slow set pectin.

9. The sleep aid according to claim 1, wherein the gel contains 85% to 90% by weight of the honey .

10. The sleep aid according to claim 1, wherein the gel contains about 0.5% by weight of the Prunus Cerasus containing melatonin precursor.

11. The sleep aid according to claim 1, wherein the gel contains 5% to 15% by weight water.

12. The sleep aid according to claim 1, wherein the gel contains about 7.5% water by weight.

13. The sleep aid according to claim 1, wherein the gel contains about 1.5% by weight of the glycerin preservative.

14. The sleep aid according to claim 1, wherein the gel contains about 0.1% by weight of the ascorbic acid antioxidant and preservative.

15. The sleep aid according to claim 1, wherein the gel contains about 0.1% by weight of the citric acid pectin activator.

16. The sleep aid according to claim 1, wherein the gel contains 0.1% to 1% by weight cherry flavor.

17. The sleep aid according to claim 1, wherein the gel contains about 0.5% by weight cherry flavor.

18. Use of the sleep aid as claimed in claim 1 to promote sleep.

19. A method comprising:
providing the sleep aid as claimed in claim 1; and
instructing a user to ingest the gel in order to promote sleep.

20. A method comprising:
obtaining the sleep aid as claimed in claim 1; and
ingesting the gel in order to promote sleep.
